(19) United States
(12) Patent Application Publication
(10) Pub. No.: US 2002/0174452 A1
(43) Pub. Date: Nov. 21, 2002

(54) Monocot seeds with increased lignan content

(76) Inventors: Norman G. Lewis, Pullman, WA (US); Laurence B. Davin, Pullman, WA (US); Ning Huang, Davis, CA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 09/944,160

(22) Filed: Aug. 30, 2001

Related U.S. Application Data
(60) Provisional application No. 60/230,632, filed on Sep. 7, 2000.

(51) Int. Cl. A01H 5/00

(52) U.S. Cl. 800/284

(57) ABSTRACT

The present invention provides methods for modifying lignan content in plants by transforming plants with vectors containing a DNA sequence encoding one or more proteins integral to the phenylpropanoid pathway leading to G-lignan formation. Such coding sequences are expressed under the control of a seed tissue specific or seed developmental stage specific promoter. Expression of the DNA sequence results in a modification of the absolute and/or relative level of an intermediate metabolite leading to the production of G-lignans (e.g., secoisolariciresinol diglucoside or matairesinol).

### CROSS-REFERENCE TO RELATED APPLICATION

This application claims benefit of priority from United States Provisional Patent Application No. 60/230,632, filed Sep. 7, 2000, under 35 U.S.C. § 119, which is incorporated herein by reference.

### FIELD OF THE INVENTION

The present invention relates to a method of increasing guaiacyl or “G” lignan content in monocot seeds, by way of the “G” lignan branch of the phenylpropanoid pathway, and to plants and seed compositions produced thereby.

### REFERENCES

Abe, et al., 1989 (rice prepro-glutelin gene, GenBank Accession No. D00584)

Adlercreutz, H., et al., Med Biol 59:259-261, 1981.

Adlercreutz, H., et al., Lancet 2:1295-9, 1982.

Adlercreutz, H., Gastroenterology 86:761-764, 1984.

Adlercreutz, H., et al., in HORMONES AND CANCER 3, Raven Press, New York, 1988.

Adlercreutz, H., Scand J Clin Lab Invest 50(Suppl 201):3-23, 1990.

Adlercreutz, H., et al., Lancet 339:1233, 1992.

Adlercreutz, H., et al., Scand J Clin Lab Invest 53:5-18, 1993.

Adlercreutz, H., et al., Lancet 342:1209-1210, 1993.

Adlercreutz, H., et al., J Steroid Biochem Mol Biol 44:147-153, 1993.

Adlercreutz, H., et al., Cancer Detect Prev 18 :259-271, 1994.

Adlercreutz, C. H. T., et al., J Nutr 125:757S-770S, 1995.

Adlercreutz et al., The Finnish Medical Society DUODECIM, Ann. Med. 29:95-120 1997.

Alber and Kawasaki, Mol. and Appl. Genet. 1:419-434, 1982.

Altschul et al., Nucl. Acids Res. 25(17) 3389-3402, 1997.

Atanassova et al., Plant Journal 8:465-477, 1995).

Ayres, D. C.; Loike, J. D. Chemistry and Pharmacology of Natural Products. Lignans. Chemical, Biological and Clinical Properties; Cambridge University Press: Cambridge, England, 1990.

Bannwart, C., et al., Clin Chim Acta 136:165-172, 1984.

Brandt et al., Carlsberg Res Commun 50:333-345, 1985.

Brandt, et al., 1985 (hordein B1 gene promoter, GenBank Accession No. X87232).

Burr and Burr, J. Mol. Biol. 154, 33-49, 1982.

Bustos, M., et al., EMBO J. 10:1469, 1991.

Davin L. B., and Lewis N. G., Plant Physiol 123(2):453-62, 2000.

Davin, L., et al., Science 275(17):362-367, 1997.

Depicker, et al., Mol. Appl. Genet. 1:561-573, 1982.

Eich, E., et al., J Med Chem 39:86-95, 1996.

Price, K. R., and Fenwick, G. R., Food Addict Contam 2:73-106, 1985.

Evans, B. A. J., et al., J Endocrinol 147:295-302, 1995.

Fotsis, T., et al., Proc Natl Acad Sci USA 90:2690-2694, 1993.

Fotsis, T., et al., J Nutr 125:S790-797, 1995.

Gielen, et al., EMBO J. 3:835-846, 1984.

Hanson, K. R. and Havir, E. A. in “The Biochemistry of Plants: A Comprehensive Treatise”, Vol. 7, Secondary Plant Products, E. E. Conn, ed., Academic Press, New York, p. 577-625, 1981.

Hrazdina, G. and Wagner, G. J., Arch. Biochem. Biophys. 237, 88-100 (1985).

Joannou et al., J Steroid Biochem Mol Biol 54: 167-184, 1995.

Jones, D. H. Phytochemistry 23, 1349-1359, 1984.

Kim and Wu, 1990 (rice glutelin gene, GenBank Accession No. X52153).

Kim, S. Y., and Wu, R., Nucl. Acids Res. 8:6845-6852, 1990.

Knutzon, D. S., et al., Proc. Natl. Acad. Sci. USA 89:2624, 1992.

Kostova, A. T., et al., J. Biol. Chem. 272(46):29473-29482, 1996.

Krol et al., Nature, 333:866-869, 1988.

Kurzer, MS et al., Cancer Epidemiol Biomarkers Prev 4:353-358, 1995.

Lam, E., and Chua, N. H., J. Biol. Chem. 266:17131, 1991.

Lampe, J W et al., Am J Clin Nutr 60:122-128, 1994.

Lampe J W et al., Cancer Epidemiol Biomarkers Prev 8(8):699-707, 1999.

Lavery et al., Genetics 94:6831-6836, 1997.

Lee et al., Proc. Nat. Acad. Sci. 88:2825, 1991.

Lewis, N. and Yamamoto, E. Annu. Rev. Plant Physiol. Plant Mol. Biol., 1990, 41, 455-496.

Li, D, et al., Cancer Lett 19;142(1):91-6, 1999.

Mazur et al., Anal Biochem 233: 169-180, 1996.

Müller and Knudsen, 1993, barley hor1-17 gene, GenBank Accession No. X60037)

Nilsson, M.; Aman, P. J. Sci. Food Agric. 0022-5142:143-148, 1997.

Nishibe, S., et al., Chem. Pharmacol. Bull. 38:1763-1765, 1990.

Mäkelä, S., et al., Environ Health Perspect 102:572-578, 1994.

Mills, P. K., et al., Cancer 64:598-604, 1989.

Montesano, R., and Orci, L., Cell 42:469-477, 1985.

Nakase, M., et al., Plant Mol. Biol. 32(4):621-630, 1996.

Okita, T. W., et al., J. Biol. Chem. 264(21):12573-12581, 1989.

Okita, et al., 1989 (rice Gt2 gene, GenBank Accession No. L36819 M28157; rice Gt3 gene, GenBank Accession No. M28158; rice Gt1 gene, GenBank Accession No. M28156; rice glutelin gene, GenBank Accession Nos. D26363, D26366, D26367, D26368 and D26369).

Pietinen, P., et al., Circulation 94:2720-2727, 1996.

Pool-Zobel B L et al., Carcinogenesis 21(6):1247-1252, 2000.

Reddy, B. S., and Cohen, L. A.(eds), in MACRONUTRIENTS AND CANCER, CRC Press, Boca Raton Fla., 1986.

Reddy, B. S., and Cohen, L. A.(eds), in MACRONUTRIENTS AND CANCER, CRC Press, Boca Raton Fla., 1986.

Rose, D. P., et al, Cancer 58:2363-2371, 1986.

Russell, et al., Transgenic Res., 6:157-168, 1997.

Schmidt et al., Science 238, 960-963, 1987.

Schroder, H. C., et al., Z. Naturforsch 45d:1215-1221, 1990.

Schubert et al., Nucl. Acids Res. 18: 377, 1990.

Setchell, K. D. R., and Adlercreutz, H., in ROLE OF THE GUT FLORA IN TOXICITY AND CANCER, Academic Press, London, 1988.

Shaw and Hannah, 1992, GenBank Accession No. S48563.

Shutt, D. A., and Cox, R. I., J. Endocrinol 52:299-310, 1972.

Stayton, M., et al., Aust. J. Plant Physiol. 18 :507, 1991.

Takaiwa, et al., 1991a (rice glutelin gene, GenBank Accession No. Y00687).

Takaiwa, et al, 1991b (rice Glu-B gene, GenBank Accession No. X54193; rice GluB-2 gene, GenBank Accession No. X54192; rice GluB-1 gene, Genbank Accession No. X54314).

Teicher, B. A., et al., Cancer Chemother Pharmacol 38:169-177, 1996.

Thompson L U, Baillieres Clin Endocrinol Metab 12(4):691-705, 1998.

Trowell, H. C., and Burkitt, D. P., in WESTERN DISEASES: THEIR EMERGENCE AND PREVENTION, Edward Arnold Ltd., London, 1981.

Whitten, P. L., and Naftolin, F., in NEW BIOLOGY OF STEROID HORMONES, Raven Press, New York, 1991.

Wolf, N., Mol. Gen. Genet. 234:33-42, 1992.

Zhang, J-X., et al., Cancer Lett 114:1-2, 1997.

### BACKGROUND OF THE INVENTION

Lignans are a large structurally diverse class of plant metabolites. Lignans exist as dimeric compounds that are found throughout the plant kingdom. A variety of biological functions and pharmacological properties have been attributed to lignans (Ayres, 1990). Lignans, in addition to neolignans and lignins, are formed in plants as part of a biochemical process known as the phenylpropanoid pathway. One of the primary products of the phenylpropanoid pathway is lignin, a three dimensional polymer which is intercalated with cellulose and hemicelluloses in the cell walls of vascular plants, to provide rigidity and compressive strength. Lignins are produced from monolignol precursors, as are lignans and neolignans.

In contrast to the polymeric structure of lignins, lignans and neolignans are dimeric compounds, typically [C6C 3] phenylpropanoid dimers, although a diversity of forms are known. While lignins are generally racemic (Lewis et al., 1990), lignans are typically optically active, existing as single enantiomeric forms, although racemic mixtures are occasionally encountered (e.g., ±syringaresinols). Lignans are usually constructed via 8.8′ carbon-carbon linkages between the phenylpropanoid subunits (Ayres et al., 1990), while neolignans are defined as being connected via linkages other than 8.8′.

A number of plant lignans of diverse structure and function are known (Ayres and Loike, 1990; Lewis et al., 1994). Examples include podophyllotoxin, a successfully employed plant anticancer agent (Ayres et al., 1990), arctigenenin and trachelogenin, which have been demonstrated to be effective against HIV (Schroder et al., 1990), and syringaresinol B-glucoside, which is reported to enhance cardiovascular activity (Nishibe et al., 1990). Plant sources of lignans include the gymnosperms (e.g., Araucariaceae, Podocarpaceae, Taxaceae, Taxodiaceae families) and the angiosperms (e.g., Magnoliiflorae, Nymphaeiflorae, Rosiflorae, Ariflorae, Commeliniflorae).

Lignans are found not only in plants, but have been identified in the biological fluids of humans and several animals as well. The two major mammalian lignans are enterolactone and enterodiol (FIG. 3). These lignans are excreted in the urine of subjects on diets rich in whole grain products, berries or fruits, and have been detected in significant levels in the plasma of subjects in geographical areas of low cancer occurrence (Adlercreutz, 1993a, 1993b; Adlercreutz, 1994). The mammalian lignans, enterolactone and enterodiol, are presumed to be formed by the metabolic action of intestinal bacteria in the gut on the plant lignans, secoisolariciresinol diglucoside and matairesinol (FIG. 3), respectively, which are released from plant food sources during digestion by the action of intestinal bacteria.

A number of positive health benefits have been associated with elevated intake of plant lignans (Adlercreutz, 1982; Adlercreutz, 1984; Adlercreutz 1990; Setchell et al., 1988 (16)), and there is evidence to support the protective role of lignans in reducing the risk of breast cancer (Adlercreutz et al., 1982; Adlercreutz et al., 1984; Bannwart et al., 1984; Setchell et al., 1988), and other hormone-related cancers and colonic cancers (Adlercreutz et al., 1995; Adlercreutz et al., 1993; Adlercreutz et al., 1988; Adlercreutz et al., 1992). Epidemiologic and in vitro studies support the role of lignans in decreasing the risk of prostate cancer (Mills et al., 1989; Zhang et al., 1997), and lignans have also been implicated in the prevention of osteoporosis. Further, phyto-oestrogens, and lignans in particular, have also been reported to reduce the risk of cardiovascular disease (Pietinen et al., 1996).

Unfortunately, the Western diet lacks foods which are lignan-rich, and the high incidence of certain major hormone-dependent cancers, colon cancer and coronary heart disease in the United States, Western Europe and Canada, when compared to countries in Asia has, in part, been attributed to the Western diet, and in particular, to a Western diet lacking certain protective compounds, i.e., lignans. (Trowell, 1981; Reddy, 1986a,b; Rose, 1986). Factors which have been attributed to low lignan levels in Western populations are (i) low intake of cereal grains, and in particular, whole grain bread, and (ii) increased production and consumption of highly processed grains, which as a result of extensive processing procedures, have greatly reduced levels of naturally occurring lignans and/or their monolignol precursors.

It has been demonstrated that increased dietary consumption of plant lignans such as those found in numerous grains results in increased levels of mammalian lignans (enterodiol and enterolactone) in the urine and feces (Joannou et al., 1995). Taken together with the positive health benefits that have been associated with elevated intake of plant lignans, consumption of plants with increased levels of lignans can result in corresponding increases in circulating levels of mammalian lignans thereby providing positive health benefits through normal dietary consumption of plants.

The prevalence of the above described diseases, particularly in Western cultures, coupled with escalating health care costs, suggests a need to develop new and aggressive approaches aimed at preventing rather than merely treating these diseases, particularly by increasing lignan intake. Ideally, this goal should be met in a manner which is readily adaptable to Western lifestyles and eating habits, does not require a drastic change or restriction in dietary intake or require consumption of dietary supplements, or adversely impact existing grain processing facilities or the quality or flavor of the resulting grains.

Flaxseed (linseed) is the most abundant source of lignans in food. Other food sources in which lignans have been detected include various grains, seeds, fruits, berries and vegetables. (Adlercreutz et al., 1997). The dietary lignans secoisolariciresinol diglucoside, isolariciresinol and lariciresinol have been demonstrated to be the immediate precursors of enterodiol, while matairesinol has been demonstrated to be the immediate precursor of enterolactone. Enterodiol, enterolactone, matairesinol and secoisolariciresinol diglucoside have been measured in human urine and feces by isotope dilution gas chromatography-mass spectrometry. (Adlercreutz et al., 1997).

### SUMMARY OF THE INVENTION

The present invention is based, in part, on the discovery that modified expression of certain enzymes integral to the phenylpropanoid pathway in plants may be effective to generate plants exhibiting modified levels and/or modified relative proportions of G-lignans (or monolignol G-lignan precursors) in plants, and in particular, in seeds of such plants.

The present invention provides methods for modifying lignan content in plants by transformin.g plants with vectors containing a DNA sequence encoding one or more proteins integral to the phenylpropanoid pathway leading to G-lignan formation. Such coding sequences are expressed under the control of a seed tissue specific or seed developmental stage specific promoter.

Expression of the DNA sequence results in a modification of the absolute and/or relative level of an intermediate metabolite leading to the production of G-lignans (e.g., secoisolariciresinol diglucoside or matairesinol).

By placing, under the control of a tissue-specific promoter, a gene sequence encoding an enzyme in the phenylpropanoid pathway at a point following coniferyl alcohol, whose expression results in production of G-lignans, or an intermediate metabolite leading to the production of G-lignans (particularly, secoisolariciresinol or matairesinol), it is possible to increase the G-lignan content specifically in seeds, or within certain seed tissues, while allowing for normal plant growth and development.

Accordingly, it is an object of the invention to provide a method of increasing the guaiacyl- (“G-”)lignan content in seeds of a monocot plant, including selecting at least one protein or enzyme integral to the pathway leading to G-lignan formation, and stably transformin.g a monocot plant with one or more chimeric gene constructs having a seed-specific transcriptional regulatory region operably linked to a nucleic acid sequence encoding at least one protein or enzyme integral to the pathway leading to G-lignan formation.

In a related aspect, at least one protein or enzyme integral to the pathway leading to G-lignan formation is selected from the group consisting of (i) a dirigent protein, (ii) pinoresinol/lariciresinol reductase, (iii) secosisolari-ciresinol dehydrogenase, and (iv) laccase.

In another related aspect, (i) a dirigent protein, (ii) pinoresinol/lariciresinol reductase, (iii) secosisolari-ciresinol dehydrogenase, and (iv) laccase, are expressed at the same time in seeds of a monocot plant.

In yet another related aspect the amount of (−)-secoisolariciresinol diglucoside or (−)-matairesinol accumulated in said seeds is greater than in seeds of an untransformed monocot plant.

In one embodiment,the seed-specific transcriptional regulatory region is derived from aleurone, pericarp, embryo or endosperm tissue. In another embodiment, the seed-specific transcriptional regulatory region is induced during seed development and corresponds to an endosperm-specifc Gt-1 promoter.

In yet another embodiment, the seed-specific transcriptional regulatory region is induced during seed development and corresponds to an aleurone-specifc Chi26 promoter.

It is another object of the invention to provide a method of stably transformin.g a monocotyledonous plant with one or more chimeric gene constructs, resulting in increased expression of the genes encoded by the chimeric gene constructs.

In one aspect, the gene in one or more chimeric gene constructs is selected from the group consisting of (i) a dirigent protein, (ii) pinoresinol/lariciresinol reductase, (iii) secosisolari-ciresinol dehydrogenase, and (iv) laccase.

In another aspect, the nucleic acid sequence encoding at least one protein or enzyme integral to the pathway leading to G-lignan formation has at least 90% sequence identity to a sequence selected from the group consisting of (i) a dirigent protein, (ii) pinoresinol/lariciresinol reductase, (iii) secosisolari-ciresinol dehydrogenase, and (iv) laccase.

In yet another aspect, the nucleic acid sequence encoding said protein or enzyme is capable of hybridizing under high stringency conditions and said protein or enzyme has substantially equivalent biological activity to the native protein or enzyme selected from the group consisting (i) a dirigent protein, (ii) pinoresinol/lariciresinol reductase, (iii) secosisolari-ciresinol dehydrogenase, and (iv) laccase.

It is another aspect of the invention to provide a transformed monocot plant produced by the method described above.

In a related aspect, the transformed monocot plant is capable of producing seeds where the amount of (−)-secoisolariciresinol diglucoside or (−)-matairesinol accumulated in the seeds of said transformed monocot plant is two or more times the amount detectable in seeds of an untransformed monocot plant.

In another related aspect, the transformed monocot plant is capable of producing seeds where the amount of (−)-secoisolariciresinol diglucoside or (−)-matairesinol accumulated in the seeds of the transformed monocot plant is five or more times the amount detectable in seeds of an untransformed monocot plant.

It is another aspect of the invention to provide a seed composition derived from a plant produced by the method described above.

In one embodiment, the seed composition includes an amount of (−)-secoisolariciresinol diglucoside or (−)-matairesinol accumulated in the seeds of said transformed monocot plant which is two or more times the amount detectable in seeds of an untransformed monocot plant.

In another embodiment, the seed composition includes an amount of (−)-secoisolariciresinol diglucoside or (−)-matairesinol accumulated in the seeds of the transformed monocot plant which is five or more times the amount detectable in seeds of an untransformed monocot plant.

It is yet another aspect of the invention to provide a library comprising seeds derived from one or more monocot plants produced by the method described above.

It is yet another aspect to provide a method of producing a progeny monocot plant by crossing one or more parent monocot plants where the amount of G-lignans in the seeds of the progeny monocot plant resulting from the crossing is greater than the amount of G-lignans in the seeds of the parent monocot plant.

In another aspect, the G-lignan enriched seed composition for use as a food additive includes a seed preparation derived from seeds of a transformed monocot plant wherein the amount of (−)-secoisolariciresinol diglucoside or (−)-matairesinol accumulated in said seeds is two or more times the amount detectable in seeds of an untransformed monocot plant.

In a related aspect, the G-lignan enriched seed composition is used as a food additive.

These and other objects and features of the invention will become more fully apparent when the following detailed description of the invention is read in conjunction with the accompanying drawings.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 represents an overview of the plant Monolignol Biosynthetic Pathway (front end of the phenylpropanoid pathway) leading to para-coumaryl alcohol, coniferyl alcohol, and sinapyl alcohol.

FIG. 2 presents an overview of the “back end” of the phenylpropanoid pathway showing the stereospecific conversion of coniferyl alcohol to secoisolariciresinol and matairesinol (i.e., the formation of G lignans).

FIG. 3 presents an overview of the metabolic conversion of the G lignans, secoisolariciresinol and matairesinol to enterodiol and enterolactone which takes place in the mammalian intestine and is mediated by intestinal bacteria, with the structures of the lignans presented in the figure as matairesinol, secoisolariciresinol diglucoside, enterolactone, and enterodiol.

FIGS. 4A and 4B presents an overview of the synthesis of deuterated d6-matairesinol (3) and d8-secoisolariciresinol (4) from matairesinol (1) (FIG. 4A); and d6-anhydro-secoisolariciresinol (5) from anhydrosecoisolariciresinol (6) (FIG. 4B).

FIGS. 5 and 11 depict images of basic cassettes for gene expression in developing rice seeds.

FIGS. 6A, 6B, 6C, and 6D present schematic depictions of the map of four plasmids used to express genes involved in lignan biosynthesis in developing rice seeds, which contain the coding sequences for various enzymes involved in lignan biosynthesis under the control of the rice endosperm-specific glutelin (Gt-1) promoter. pGt-1-DIRG (FIG. 6A) contains the coding sequence for dirigent protein; pGt-1-LACC (FIG. 6B) contains the coding sequence for laccase; pGt-1-REDS (FIG. 6C) contains the coding sequence for pinoresinol/lariciresinol reductase; and pGt-1-DEHY (FIG. 6D) contains the coding sequence for secosisolariciresinol dehydrogenase.

FIGS. 7A, 7B, and 7C dipict the nucleotide sequence of plasmid pAPI245 used to express laccase.

FIGS. 8A, 8B, and 8C depict the nucleotide sequence of plasmid pAPI244 used to express dirigent.

FIGS. 9A, 9B, and 9C depict the nucleotide sequence of plasmid pAPI246 used to express pinoresinol/lariciresinol reductase.

Figs 10A, 10B, and 10C depict the nucleotide sequence of plasmid pAPI249 used to express secosisolariciresinol dehydrogenase.

FIG. 12 presents a flow diagram of the rice transformation procedure where an expression vector is introduced into callus tissue, which is selected, then regenerated to produce transgenic seedlings, which are grown to transgenic R₀ plants that produce transgenic R₁ seed.

FIG. 14 is a graphic depiction of the results of an analysis of Matairesinol in numerous transgenic rice lines transformed with four vectors containing four lignan genes, relative to untransformed control M202 and TP309 plants.

FIG. 13 is a schematic depiction of an exemplary procedure used to detect lignan production in half seeds of transgenic rice, as a part of the process of selecting transgenic plants for further propagation in the greenhouse of field.

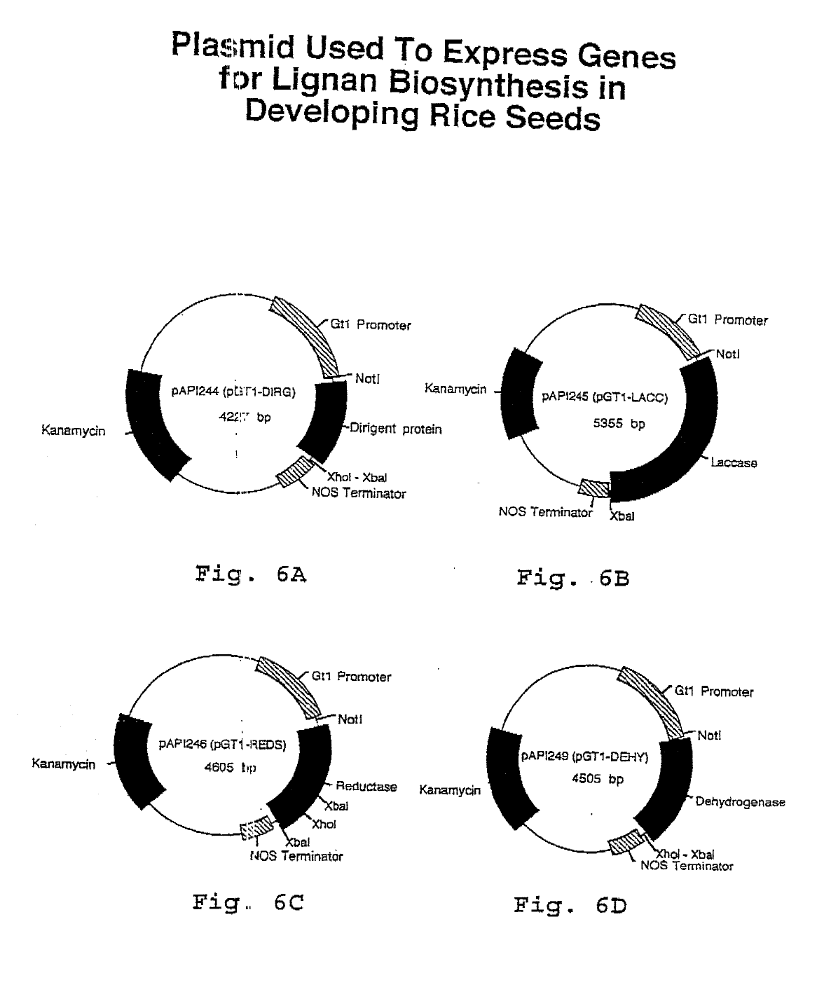

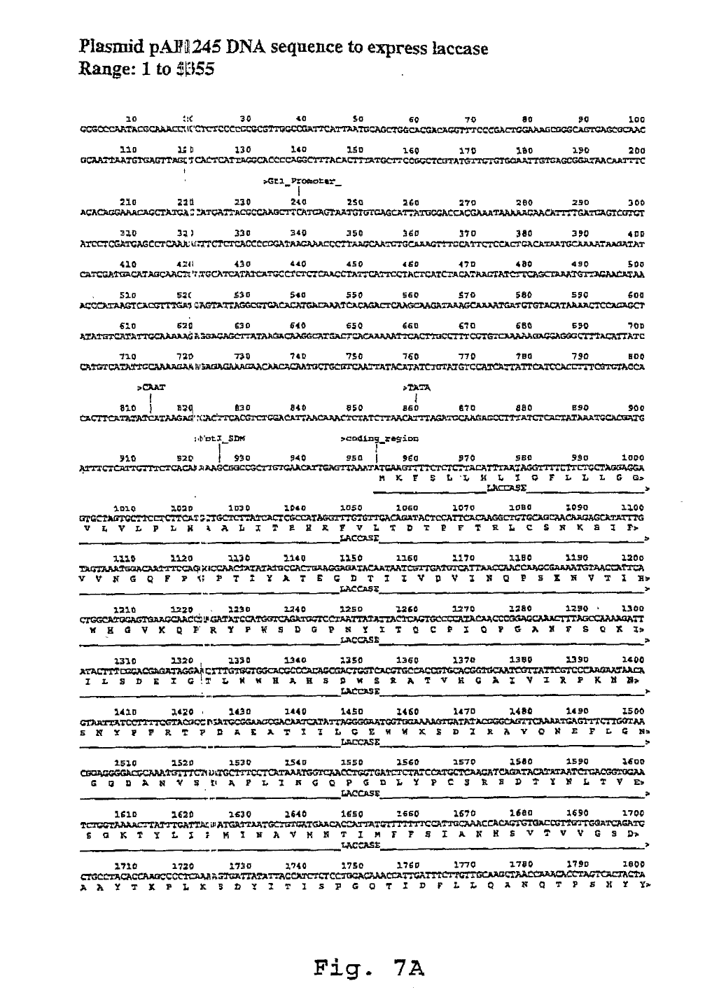

FIG. 15 depicts an image of an ethidium bromide stained gel showing the results of electrophoresis of PCR products derived by amplification of DNA derived from rice plants transformed with the four heterologous nucleic acid constructs presented in FIGS. 6A, 6B, 6C, and 6D, DNA from non-transformed plants, or control DNA from reductase, laccase, dehydrogenase and dirigent protein encoding DNA constructs, by performin.g PCR using primers specific to the reductase, laccase, dehydrogenase and dirigent protein coding sequences at the same time.

### DETAILED DESCRIPTION OF THE PREFERRED EMBODIMENT

I. DEFINITIONS

Unless otherwise indicated, all terms used herein have the same meaning as they would to one skilled in the art of the present invention. Practitioners are particularly directed to Sambrook et al. (1989) Molecular Cloning: A Laboratory Manual (Second Edition), Cold Spring Harbor Press, Plainview, N.Y. and Ausubel F M et al. (1993) Current Protocols in Molecular Biology, John Wiley & Sons, New York, N.Y., for definitions and terms of the art. It is to be understood that this invention is not limited to the particular methodology, protocols, and reagents described, as these may vary.

“Chimeric gene” or “heterologous nucleic acid construct”, as defined herein refers to a non-native gene (i.e., one which has been introduced into a host) which may be composed of parts of different genes, including regulatory elements. A chimeric gene construct for plant/seed transformation is typically composed of a transcriptional regulatory region (promoter) operably linked to a heterologous protein coding sequence, or, in a selectable-marker chimeric gene, to a selectable marker gene encoding a protein conferring antibiotic resistance to transformed plant cells. A typical chimeric gene of the present invention, for transformation into a plant, may include a transcriptional regulatory region inducible during seed development, a protein coding sequence, and a termin.ator sequence. A chimeric gene construct may also include a second DNA sequence encoding a signal peptide if secretion of the target protein is desired.

Monocot” or “monocotyledonous plant”, refers to an angiosperm (i.e., a flowering plant) that has only a single cotyledon (seed leaf) that is formed during embryogenesis. Exemplary monocots include wheat, maize, barley, rice, millet, oats, rye, triticale, sorghum, and corn.

“Seed” is meant to encompass all seed components, including, for example, the coleoptile and leaves, radicle and coleorhiza, scutulum, starchy endosperm, aleurone layer, pericarp and/or testa, either during seed maturation and seed germin.ation.

A “phenylpropanoid” compound is a compound having a 3 carbon side chain on an aromatic ring—a common structural element shared by all of the metabolites in the monolignol pathway.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

By “promoter” or “transcriptional or translational regulatory region” is meant a sequence of DNA that directs or regulates transcription of a downstream gene heterologous to the promoter, and includes promoters derived by means of ligation with operator regions, random or controlled mutagenesis, addition or duplication of enhancer sequences, addition or modification with synthetic linkers, and the like.

The transcriptional and translational regulatory nucleic acid will generally be appropriate to the host cell used to express the target protein; for example, transcriptional and translational regulatory nucleic acid sequences from specific plant tissues are preferably used to express the protein (e.g. enzyme) intermediates of the phenylpropanoid pathway protein in various seed tissues. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences. Specific types of promoters are defined below.

Suitable sources for gene sequences useful in the present invention are dicot or monocot plants, or bacteria or fungi.

“Seed development” refers to any seed condition, from fertilization to late-stage germin.ation, characterized by induction of one or more enzymes in the seed. Seed development refers to both seed maturation and seed germin.ation.

A “seed developmental stage specific promoter” or “seed inducible promoter” is one which is inducible in seeds, generally at particular stages of seed development, either under the control of endogenous factors present in the seed, plant hormones, such as abscissic or gibberellic acid, or physical stimuli, e.g. , heat and moisture. A seed-induced promoter may be “seed-specific”, meaning it is induced preferentially in seeds relative to other tissue (Knutzon et al., 1992; Bustos et al., 1991; Lam et al., 1991; and Stayton et al., 1991), “seed tissue specific”, meaning it is induced preferentially in certain seed cells, e.g., testa-layer cells, or “seed-stage-specific”, meaning it is induced during certain stages of seed maturation or germin.ation.

“Stably transformed” as used herein refers to a plant cell or plant or seed that has foreign nucleic acid integrated into its genome which is maintained through at least two or more generations.

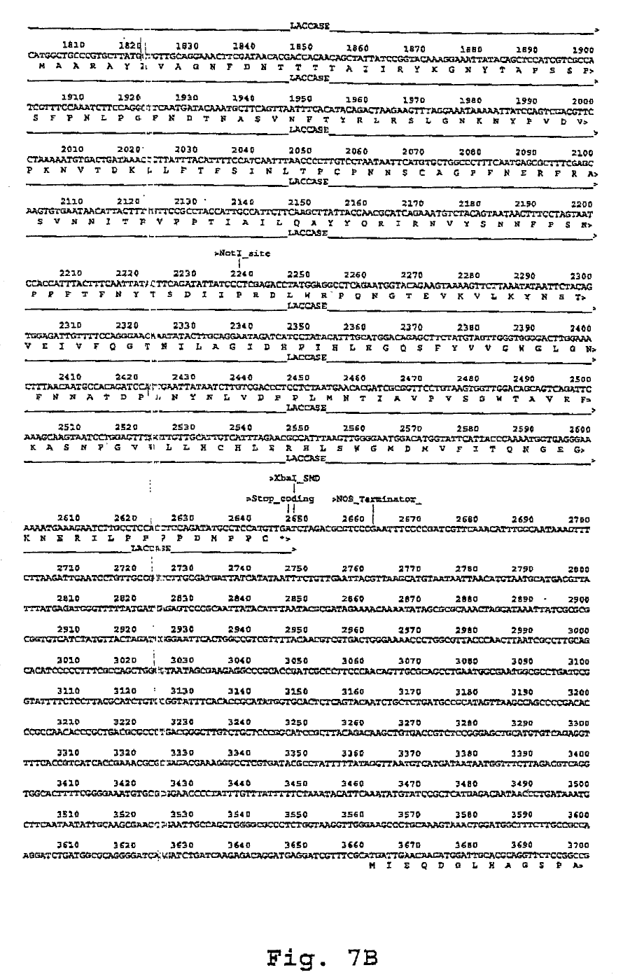

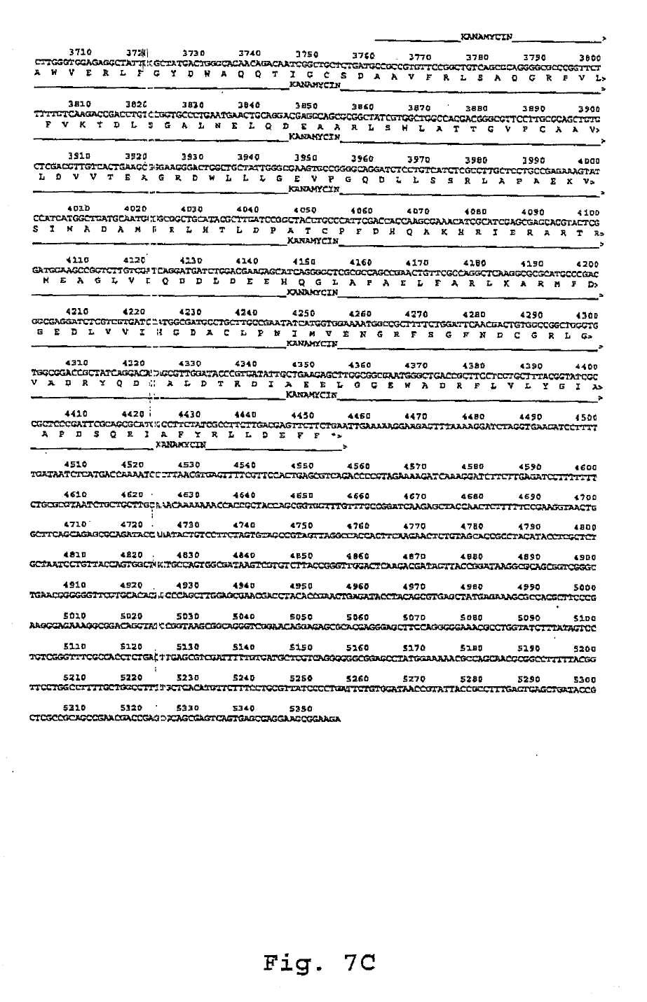

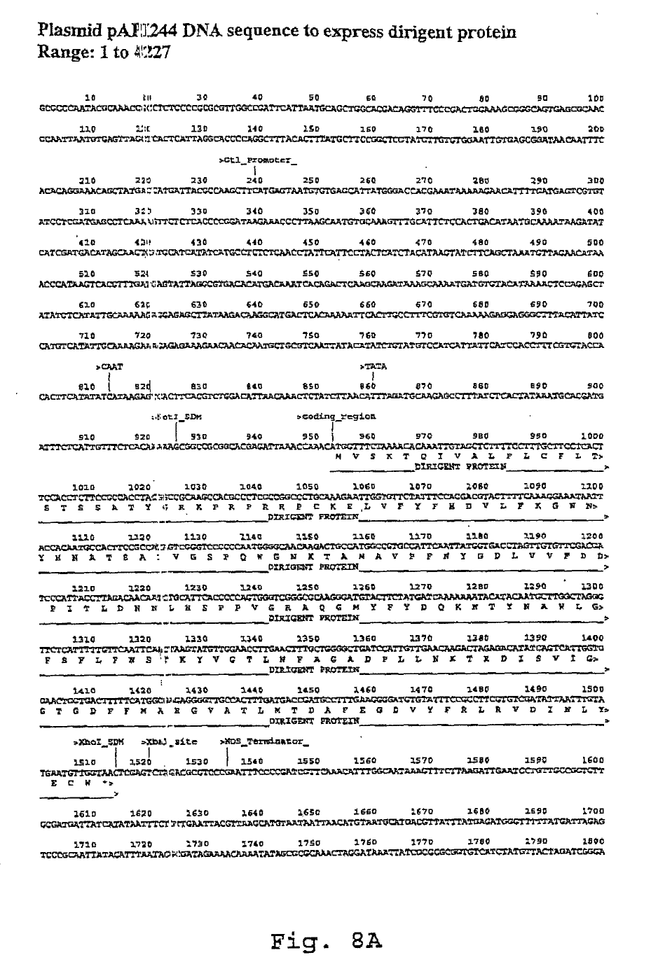

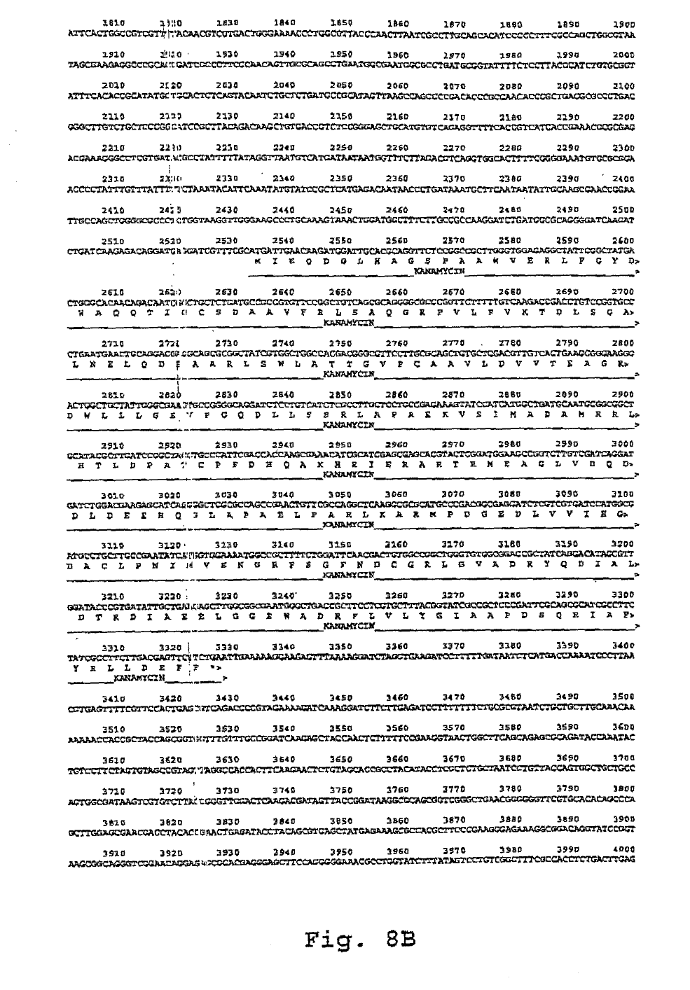

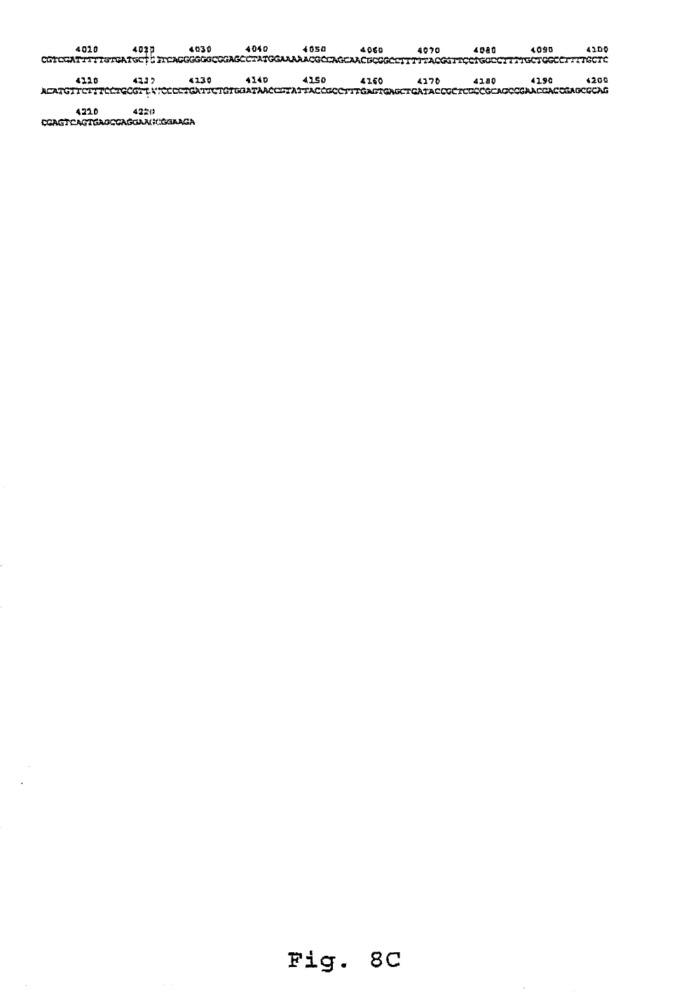

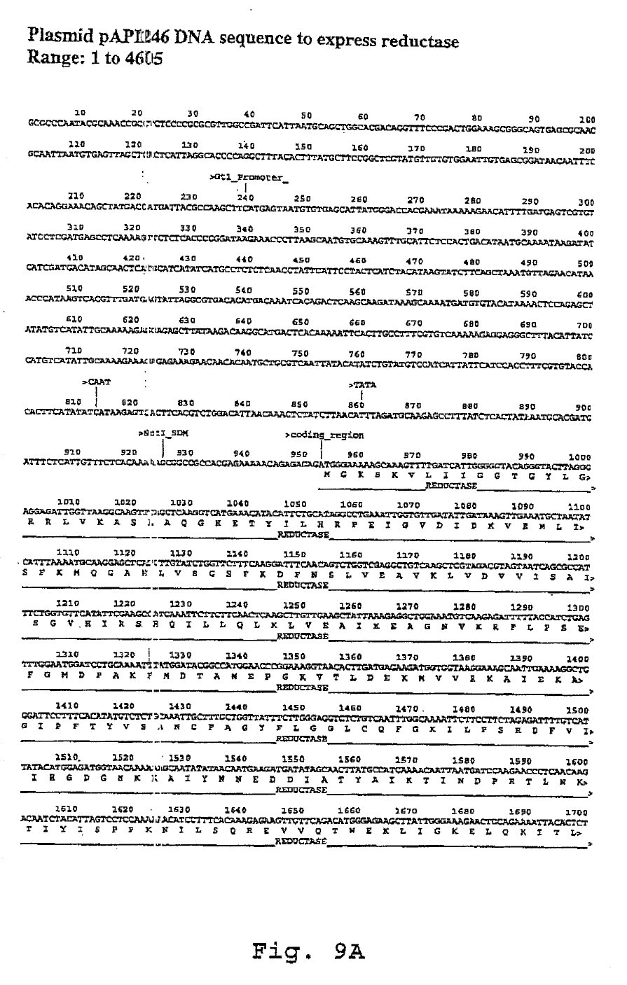

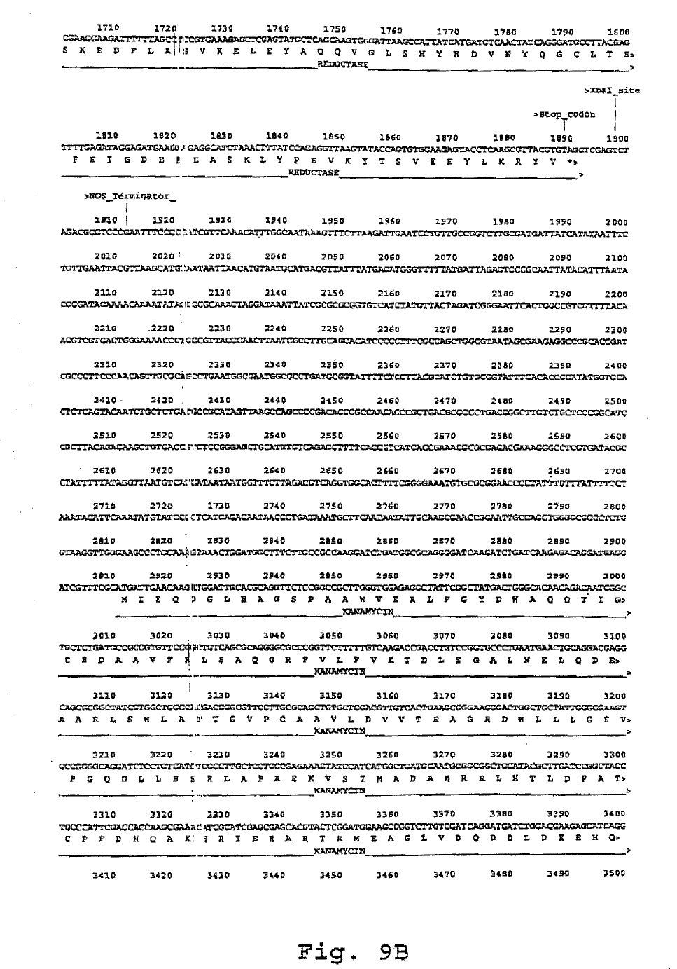

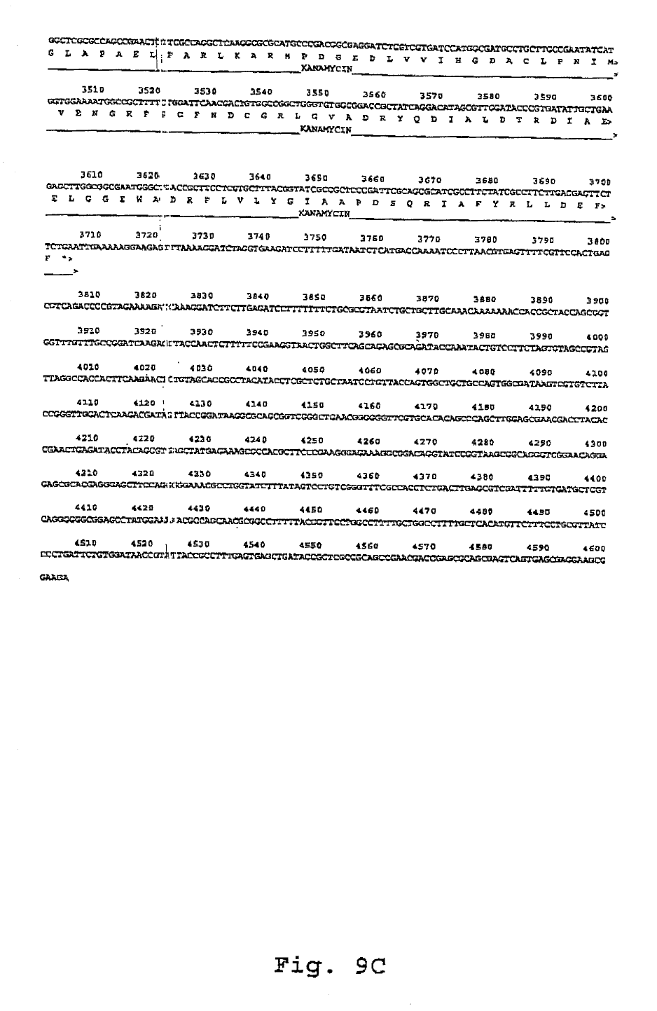

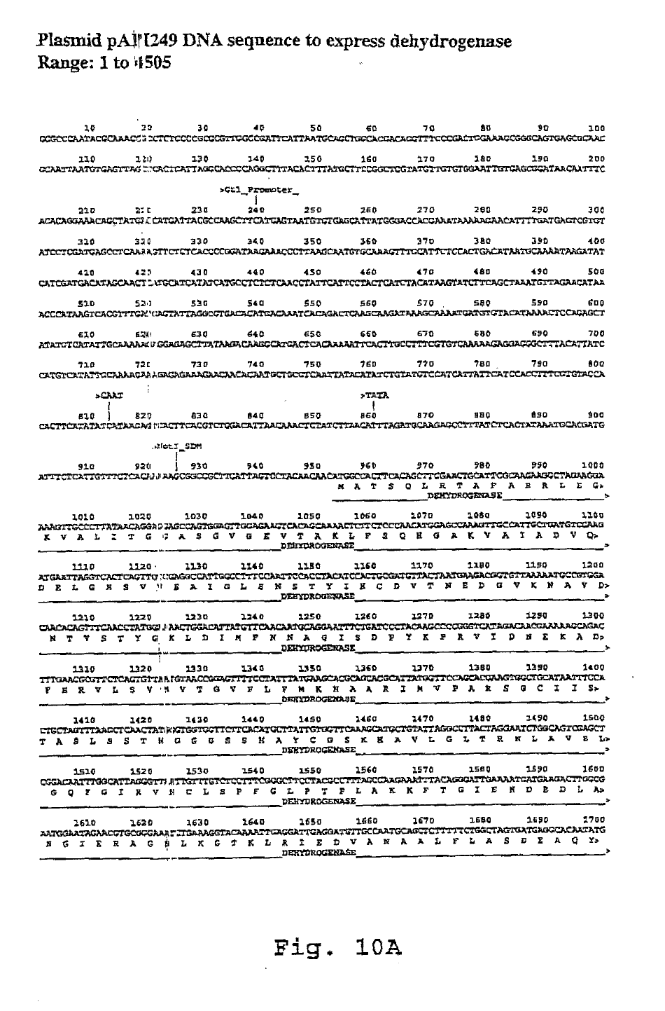

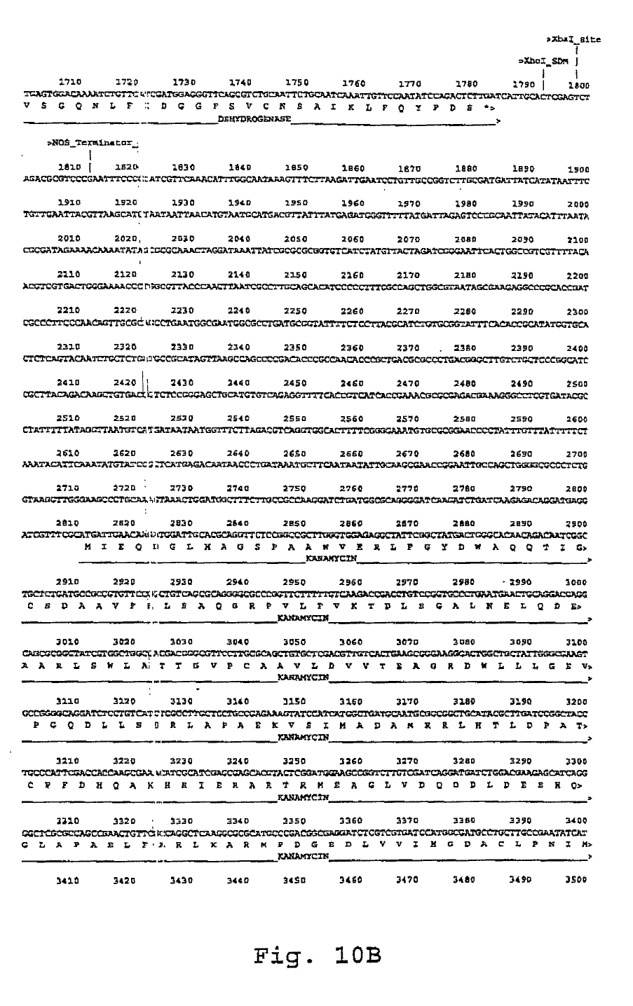

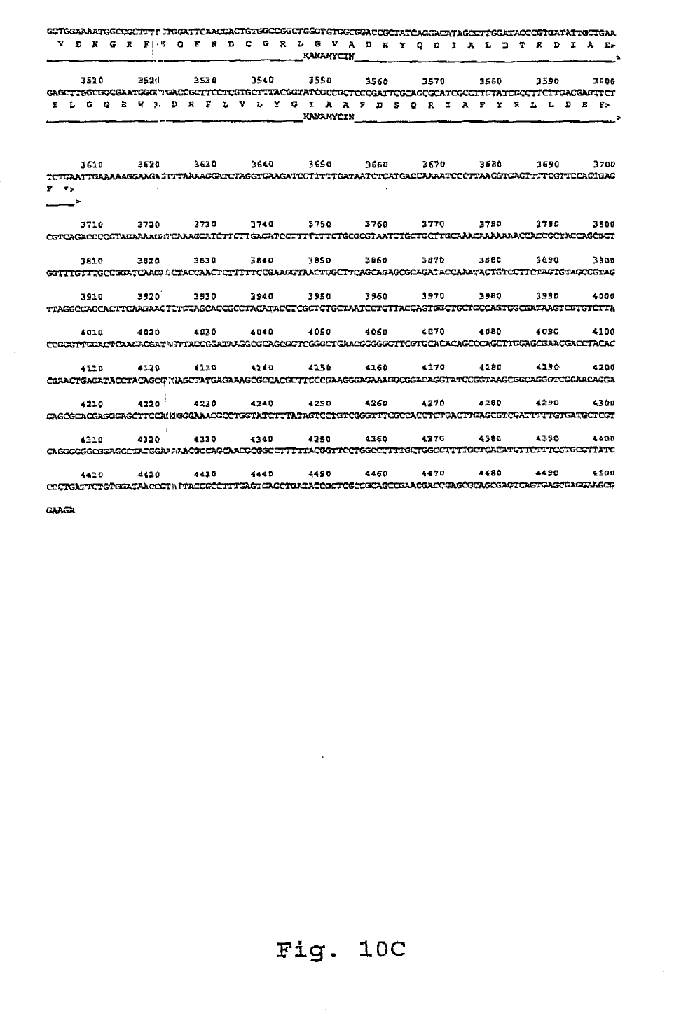

A “gene encoding a protein” refers to DNA encoding a protein having a specified function in plant cells. Examples include the representative enzyme-coding sequences disclosed herein (e.g., a gene or gene family encoding an enzyme or protein which functions in the plant phenylpropanoid pathway, including laccase (SEQ ID NO: 11; FIGS. 7A, 7B, and 7C), a dirigent protein (SEQ ID NO: 15; FIGS. 8A, 8B, and 8C), for pinoresinol/lariciresinol reductase (SEQ ID NO: 19; FIGS. 9A, 9B, and 9C), and secosisolariciresinol dehydrogenase (SEQ ID NO: 23; FIGS. 10A, 10B, and 10C), and sequences have appropriate levels of sequence identity to or capable of hybridizing under high stringency conditions to those described herein.

A “native protein or enzyme” means a protein and enzyme represented by the amin.o acid sequence and in the form endogenous to the plant tissue in which it is found in nature. Such a protein or enzyme is encoded by the nucleic acid sequences which encodes the native protein and has a biological activity as it is found in nature. An example of such a native enzyme is laccase found in docot or monocot plants, having the amin.o acid sequence set forth in SEQ ID NO: 12, encoded by the nucleic acid sequence set forth in SEQ ID NO: 11, and which functions together with dirigent protein to facilitate the stereospecific conversion of E-coniferyl alcohol to the 8,8′-linked dimeric lignan, (+)-pinoresinol.

A “protein or enzyme variant” means a protein or enzyme which comprises the biological activity of the native protein or enzyme and is further defined as having at least about 80% amin.o acid sequence identity with the “native protein or enzyme”. Such a “protein or enzyme variant” includes, for example, the amin.o acid sequence for laccase wherein one or more amin.o acid residues are added, or deleted, at the N or C ternin.us of the sequence, or substituted for one or more amin.o acid residues within the sequence of SEQ ID NO: 12. Ordinarily, a “protein or enzyme variant” will have at least about 80% amin.o acid sequence identity, preferably at least about 85% amin.o acid sequence identity, more preferably at least about 90% amin.o acid sequence identity and even more preferably at least about 95 or 98% amin.o acid sequence identity with the amin.o acid sequence of the native protein or enzyme.

As used herein, the term “sequence identity” means nucleic acid or amin.o acid sequence identity in two or more aligned sequences, aligned using a sequence alignment program. Sequence searches are preferably carried out using the BLASTN program when evaluating the of a given nucleic acid sequence relative to nucleic acid sequences in the GenBank DNA Sequences and other public databases. The BLASTX program is preferred for searching nucleic acid sequences which have been translated in all reading frames against amin.o acid sequences in the GenBank Protein Sequences and other public databases. Both BLASTN and BLASTX are run using default parameters of an open gap penalty of 11.0, and an extended gap penalty of 1.0, and utilize the BLOSUM-62 matrix. [See, Altschul et al., 1997.]

A preferred alignment of selected sequences in order to determin.e “% identity” between two or more sequences, is performed using the CLUSTAL-W program in MacVector, operated with default parameters, including an open gap penalty of 10.0, an extended gap penalty of 0.1, and a BLOSUM 30 similarity matrix.

A nucleic acid sequence is considered to be “selectively hybridizable” to a reference nucleic acid sequence if the two sequences specifically hybridize to one another under high stringency hybridization and wash conditions. A nucleic acid sequence is considered to be “selectively hybridizable” to a reference nucleic acid sequence if the two sequences specifically hybridize to one another under moderate to high stringency hybridization and wash conditions. Hybridization conditions are based on the melting temperature (Tm) of the nucleic acid binding complex or probe. For example, “maximum stringency” typically occurs at about Tm-5° C. (5° below the Tm of the probe); “high stringency” at about 5-10° below the Tm; “intermediate stringency” at about 10-20° below the Tm of the probe; and “low stringency” at about 20-25° below the Tm. Functionally, maximum stringency conditions may be used to identify sequences having strict identity or near-strict identity with the hybridization probe; while high stringency conditions are used to identify sequences having about 80% or more sequence identity with the probe.

High stringency hybridization conditions are well known in the art (see, for example, Sambrook, et al. (1989) Chapters 9 and 11, and in Ausubel, F. M., et al., 1993, expressly incorporated by reference herein). An example of high stringency conditions includes hybridization at about 42° C. in 50% formamide, 5×SSC, 5×Denhardt's solution, 0.5% SDS and 100 μg/ml denatured carrier DNA followed by washing two times in 2×SSC and 0.5% SDS at room temperature and two additional times in 0.1×SSC and 0.5% SDS at 43° C.

As used herein, the term “expression” refers to the process by which a polypeptide is produced based on the nucleic acid sequence of a gene. The process includes both transcription and translation.

As used herein, the terms “transformed”, “stably transformed” or “transgenic” with reference to a plant cell means the plant cell has a non-native (heterologous) nucleic acid sequence integrated into its genome which is maintained through two or more generations.

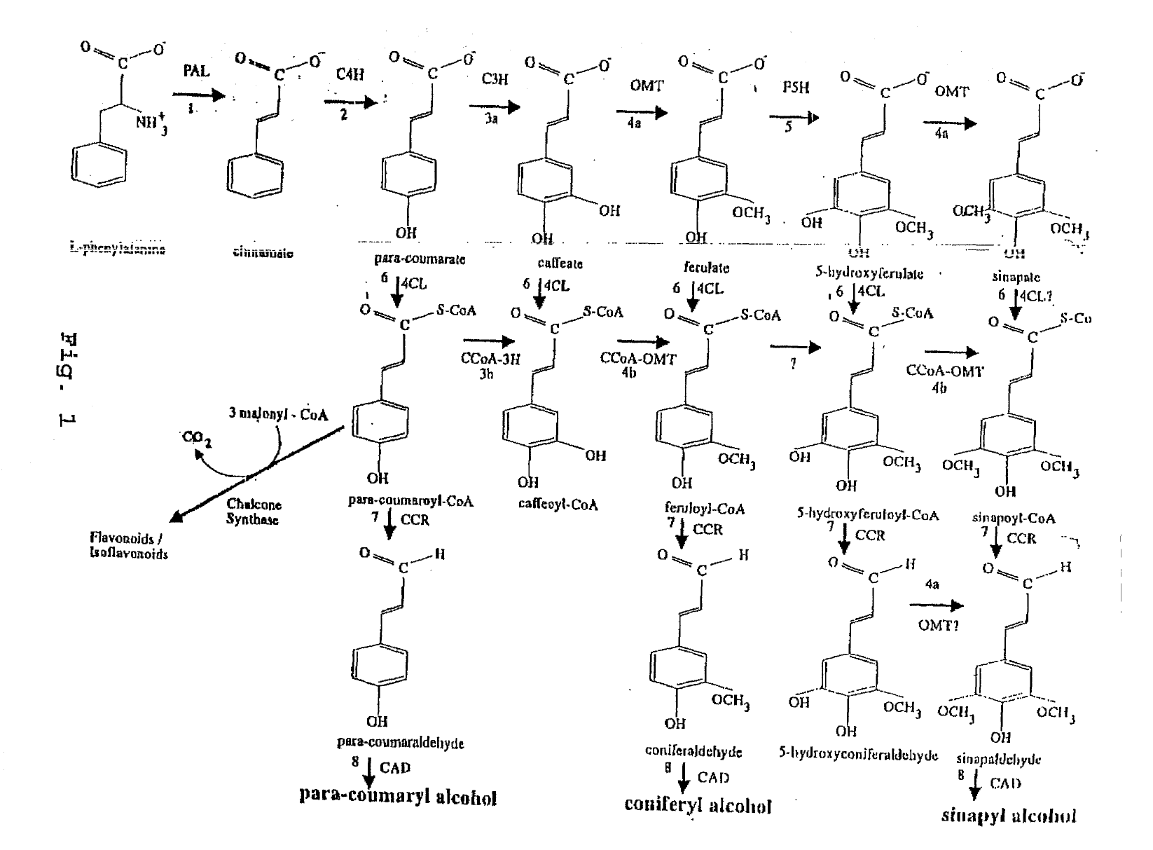

“G-lignans”, as referred to herein, are lignans derived from coniferyl alcohol or its precursors, as illustrated in FIG. 1. The G-lignans are formed from coniferyl alcohol in a series of bimolecular radical coupling reactions, via a branch point in the biosynthetic pathway which leads to G-lignans rather than to H or S lignans, or the polymeric metabolite of any one of G, H or S lignans, namely, lignins. Preferred G-lignans include (+)-pinoresinol, (+)-lariciresinol, (−) secoisolariciresinol-isolariciresinol, (−) secoisolariciresinol diglucoside, and (−)-matairesinol, that is, those lignans derived from coniferyl alcohol up to and including matairesinol.

II. LIGNAN PATHWAYS

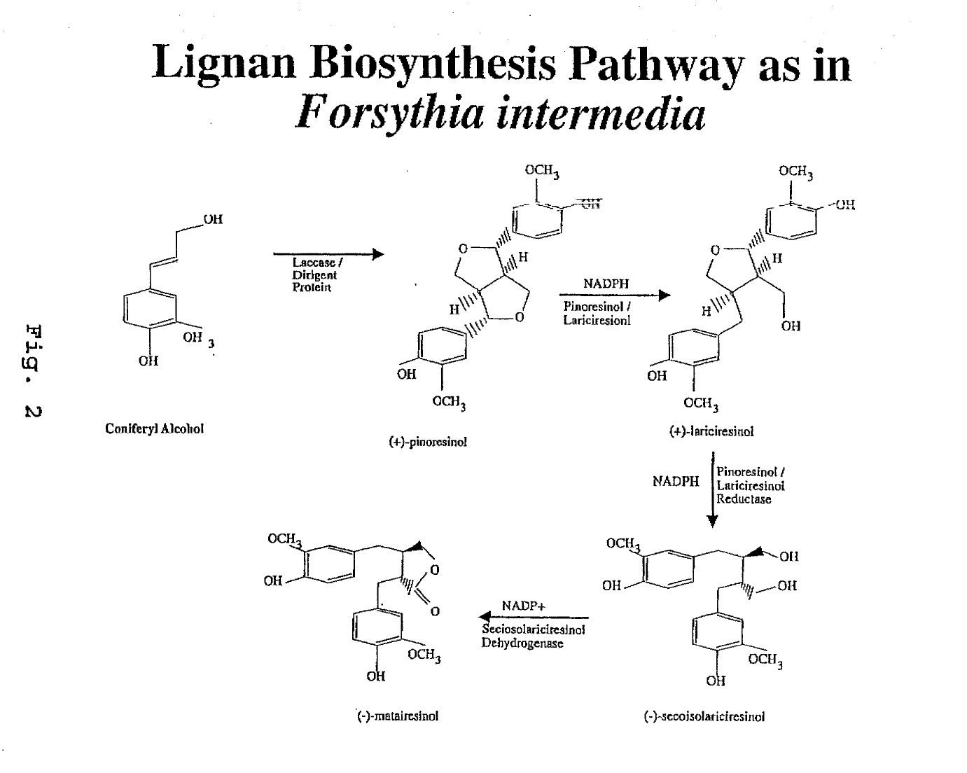

The plant biochemical pathways leading to E-coniferyl alcohol has been described as the “front end” of the plant phenylpropanoid pathway (FIG. 1) and the pathway from E-coniferyl alcohol to the lignans has been designated the “back end” of the plant phenylpropanoid pathway (FIG. 2). The present invention is directed to modifying the phenylpropanoid pathway in monocots, particularly G-lignan expression in seeds, by altering the expression of various genes which regulate levels of lignan precursors and lignans, suitable for expression in developing, maturing or germin.ating seeds as further described below.

A. THE MONOLIGNAN BIOSYNTHETIC PATHWAY-FRONT END

The plant biochemical pathway leading to coniferyl alcohol (and other monolignols as well) is referred to as the Monolignol Pathway, based on conversion involving non-dimeric phenylpropanoid monomers. The Monolignol Pathway is part of a series of enzyme-catalyzed reactions in plants referred to as phenylpropanoid metabolism, which is utilized by plants to synthesize a number of different secondary products or metabolites.

In this multistep process, phenylalanine is converted to coniferyl alcohol and two other monolignols, para-coumaryl alcohol and sinapyl alcohol, as shown in FIG. 1. The Monolignol Pathway is the starting point for formin.g not only monolignols, but also for formin.g lignins, lignans, flavenoids, isoflavenoids, coumarins, soluble esters and stilbenes, whose formation also proceeds through at least a portion of this shared pathway.

As indicated in FIG. 1, upon formation of each of the three monolignols, these compounds may either proceed to form dimeric lignans or may go on to polymerize to form lignin. The three monolignols each give rise to different types of lignin. Polymerization of para-coumaryl alcohol subunits leads to formation of H-lignin, polymerization of coniferyl alcohol leads to formation of G-lignin, and polymerization of sinapyl alcohol leads to formation of S-lignin. The names of each of the lignan pathways as used herein correspond to the termin.ology used to indicate lignan type. Thus, lignans derived from coumaryl alcohol are referred to herein as H-lignans, lignans derived from coniferyl alcohol are referred to as G-lignans, and lignans derived from sinapyl alcohol are referred to as S-lignans. Pathways competing with the G-lignan pathway are discussed herein primarily in the context of their impact on the formation of G-lignans.

A summary of each of the pertinent enzymes in the Monolignol Pathway and the enzymes necessary for converting phenylalanine to each of the monolignols, is provided in below.

A1. PHENYLALANINE AMMONIA LYASE

Phenylalanine ammonia-lyase, (PAL), is the enzyme which catalyzes the first reaction in the general phenylpropanoid pathway, and is often referred to as the entry point into plant phenylpropanoid metabolism. PAL catalyzes the deamin.ation of phenylalanine to form cinnamate and ammonia (which is most likely recaptured within the plant), as indicated in FIG. 1.

PAL exists as a tetramer in vascular plants (Hanson and Havir, 1981; Jones, 1984), and PAL subunits are typically encoded by multigene families in angiosperms, depending on the species. Coding sequences for PAL and other plant enzymes described herein are discussed in greater, below.

A2. CINNAMATE 4-HYDROXYLASE

The enzyme, cinnamate 4-hydroxylase (C4H), is a cytochrome P-450-linked monooxygenase which functions to hydroxylate cinnamate at the para ring position, to form the mono-hydroxylated compound, para-coumarate. The enzyme acts by cleaving molecular oxygen, followed by addition of one oxygen atom to the aromatic ring and reduction of the other oxygen atom to water. It has been suggested that the substrate cinnamate is transferred to C4H from PAL by a process known as metabolic channeling (Hrazdina and Wagner, 1985), a mechanism in which intermediates in a metabolic pathway are transferred between enzymes via a multi enzyme complex.

A3. CAFFEATE O-METHYLTRANSFERASE

Caffeate O-Methyltransferase (C-OMT) catalyzes the methylation of the 3-hydroxy group of caffeic acid to form the corresponding 3-methoxy compound, ferulic acid. The C-OMT-promoted methylation reaction limits the reactivity of the 3-hydroxy group, thereby reducing the number of sites on the aromatic ring that can form bonds to other monolignols.

C-OMT utilizes S-adenosylmethionine to provide the methyl donor group, and this same enzyme is also believed to catalyze the methylation of 5-hydroxyferulate to sinapate, as indicated in FIG. 1. Thus, C-OMT catalyzes reactions leading to the formation of both coniferyl and sinapyl alcohols, with the preference for substrate differing among different plant species (Shumada et al., 1973; Kuroda et al., 1981).

A4. FERULATE 5-HYDROXYLASE

Ferulate 5-hydroxylase, (F5H), is another cytochrome P450-linked monooxygenase, functioning to hydroxylate ferulate to the corresponding dihydroxy, methoxy-substituted aromatic compound, 5-hydroxy ferrulate. The reaction catalyzed by this enzyme represents a branch point in the monolignol pathway, leading to formation of non-G lignan products, and more specifically, to sinapyl alcohol derivatives (i e. S-lignans).

A5. 4-COUMARIC ACID COENZYME LIGASE

The enzyme 4-coumaric acid coenzyme ligase (4Cl) catalyzes the formation of CoA thioesters of cinnamic acids in the biosynthesis of a wide variety of phenolic derivatives, including benzoic acid, condensed tannins, flavenoids, and the cinnamyl alcohols. The activity of 4Cl is dependent upon ATP, and thioester formation proceeds through an intermediate acyl adenylate which reacts with CoA to form the thioester (Whetten et al., 1995). As indicated in FIG. 1, 4Cl catalyzes formation of CoA thioesters from the substrates para-coumarate, caffeate, ferulate, and 5-hydroxyferulate, and sinapate and thus contributes to formin.g each of the monolignols, para-coumaryl alcohol, coniferyl alcohol, and sinapyl alcohol.

A6. CHALCONE SYNTHASE

Chalcone synthase, (CHS), represents the initial enzyme for entry into the flavenoid pathway, at a branch point of the phenylpropanoid pathway which leads, not to lignans or lignins, but to flavenoid compounds composed of a basic 15-carbon skeleton.

Specifically, chalcone synthase (CHS) catalyzes the production of a chalcone via condensation of three molecules of malonyl-Coenzyme A and one molecule of para-coumaroyl-Coenzyme A. The ultimate products of this biosynthetic pathway are anthocyanins, glycosylated flavenoid pigments which play many diverse roles in plants.

The reaction of para-coumaroyl-CoA with CHS represents a branch point in the phenylpropanoid pathway, leading to formation of non-G lignan products. In fact, the substrate para-coumaroyl-CoA can react with three different enzymes, with the hydroxylation reaction catalyzed by CCoA-3H leading to the eventual formation of the monolignols, coniferyl and sinapyl alcohol, and the reduction catalyzed by cinnamoyl-Coenzyme A reductase (CCR, described in section A.9 below) leading to formation of the third monolignol, para-coumaryl alcohol.

A7. COUMARYL-COA 3-HYDROXYLASE

Coumaryl-CoA 3-hydroxylase, (CCoA-3H) is an enzyme which also acts upon the substrate, para-coumaroyl CoA, facilitating incorporation of a hydroxyl group at the 3 position of the aromatic ring to form the corresponding dihydroxy compound, caffeoyl-CoA. The intermediate formed by action of this enzyme, caffeoyl-CoA, is a precursor to a metabolite which provides direct entry into the G-lignan pathway.

A8. CAFFEOYL-COENZYME A O-METHYLTRANSFERASE

Caffeoyl-Coenzyme A O-Methyltransferase, (CCoA-OMT), plays a role in methylating both caffeoyl-CoA and 5-hydroxyferuloyl-CoA, thereby contributing to formation of both coniferyl and sinapyl alcohol and their corresponding downstream lignan products.

A9. CINNAMOYL-COENZYME A REDUCTASE

The enzyme, cinnamoyl-Coenzyme A reductase, (CCR), is a non-specific enzyme which catalyzes the reduction of hydroxycinnamoyl-CoA thioesters (e.g., para-coumaroyl-CoA, feruloyl-CoA, 5-hydroxyferuloyl-Coa, and sinapoyl-CoA) to the corresponding aldehydes. In general, this enzyme does not exhibit much specificity for one thioester substrate over another, although feruloyl-CoA is reported to be the best substrate for CCR in soybean (Wengenmayer et al., 1976), with the product of this reaction (i.e., coniferaldehyde) being the immediate precursor of coniferyl alcohol.

A10. CINNAMOYL ALCOHOL DEHYDROGENASE

The final step in the Monolignol Pathway, i.e., the reduction of hydroxycinnamaldehydes to hydroxycinnamyl alcohols (the monolignols), is catalyzed by cinnamoyl alcohol dehydrogenase, (CAD). In addition to regulation by plant developmental pathways, CAD is expressed in response to stress (Galliano et al, 1983, Campbell et al., 1992a) and wounding. In contrast to CCR, CAD has been reported to display different substrate affinities depending upon the plant species, although in general, angiosperm CAD preparations tend to exhibit equal activities with coniferaldehyde and sinapylaldehyde (Gross, 1985).

B. THE PLANT “G-LIGNAN” PATHWAY

The back end of the G-lignan pathway involves a series of reactions subsequent to monolignol formation, originating with the monolignol, coniferyl alcohol. An overview of the G-lignan pathway, and in particular, reactions pertinent to the formation of matairesinol and secoisolariciresinol, are shown in FIG. 2.

Once formed, coniferyl alcohol can react via two different metabolic pathways. One pathway, the lignin pathway, leads to formation of lignins via oxidation of coniferyl alcohol, followed by a series of coupling reactions which ultimately give rise to the macromolecular racemic G-lignins. Lignins may contain mixtures of one or more of H, G and S monolignol subunits. The various types of lignins are characterized by the different relative amounts of the monolignol subunits, e.g. G-S lignins or S-lignins.

An alternative pathway, the G-lignan pathway, involves stereoselective dimerization of coniferyl alcohol to form the lignan, (+)pinoresinol, followed by subsequent stereospecific conversions of (+)pinoresinol to form various optically active G-lignans [(+)lariciresinol, (−)secoisolariciresinol, (−) secoisolariciresinol-isolariciresinol diglucoside and (−)matairesinol). Although not shown, further metabolism of matairesinol presumably affords lignans such as (−)trachelogenin and (−)podophyllotoxin, and also affords entry into various lignan subclasses.

Proteins and enzymes specific to the biosynthesis of G-lignans, (i.e., those which act specifically on coniferyl alcohol or its lignan metabolites), and those at branch points in the phenylpropanoid pathway leading to competing pathways, find utility in the methods described herein.

The sequential conversion of E-coniferyl alcohol to (+)pinoresinol, (+)lariciresinol, (−)secoisolariciresinol, (−)secoisolariciresinol diglucoside, and finally (−)matairesinol is facilitated by dirigent protein, pinoresinol/lariciresinol reductase, secoisolariciresinol diglucosyl transferase, and secoisolariciresinol dehydrogenase, respectively.

B1. “DIRIGENT PROTEIN” (DIRG)

The first step in the G-lignan pathway is the stereospecific conversion of E-coniferyl alcohol to the 8,8′-linked dimeric lignan, (+)-pinoresinol. The reaction is facilitated by a stereospecific coupling agent referred to as a “dirigent protein”, (Lewis et al., WO 98/20113), meaning a protein which aligns or guides (Davin et al., 1997). The enzyme has been characterized as a 78-kD protein lacking a catalytically active oxidative center. The dirigent protein serves to bind and orient two coniferyl alcohol-derived free radicals arising from a one electron oxidation (typically resulting from action of an oxidase), which then undergo stereoselective coupling to form (+)-pinoresinol. In model in vitro studies, the dirigent protein was shown to possess marked substrate specificity for E-coniferyl alcohol over sinapyl and para-coumaryl alcohols (Davin et al., 1997), making this protein a preferred target for up-regulation in monocot seeds according to the methods described herein.

B2. LACCASE (LACC)

The first step in the G-lignan pathway is the stereospecific conversion of E-coniferyl alcohol to the 8,8′-linked dimeric lignan, (+)-pinoresinol. The reaction is facilitated by dirigent protein, together with laccase.

B3. PINORESINOL/LARICIRESINOL REDUCTASE (REDS)

The sequential conversion of (+)pinoresinol into (+)lariciresinol and (−)-secoisolariciresinol is facilitated by the enzyme, pinoresinol/lariciresinol reductase. Two isoforms of the reductase have been isolated from F. intermedia (Kostova, et al., 1996; Lewis, et al., WO 98/20113), and both act to catalyze the sequential reduction of (+)pinoresinol to (+)lariciresinol, and (+)lariciresinol to (−)-secoisolariciresinol, i.e., each isoform is capable of catalyzing the reaction of both substrates. The products of these reactions, lariciresinol and secoisolariciresinol, can be either (+) or (−). Both isoforms have comparable molecular masses of ˜34.9 kD.

B4. SECOISOLARICIRESINOL DEHYDROGENASE (DEHY)

The conversion of (−)-secoisolariciresinol into (−)-matairesinol is facilitated by the enzyme, secoisolariciresinol dehydrogenase.

B5. SECOISOLARICIRESINOL DIGLUCOSYL TRANSFERASE

The conversion of (−)-secoisolariciresinol into (−)-secoisolariciresinol diglucoside is facilitated by the enzyme, secoisolariciresinol diglucosyl transferase.

III. MODIFIED G-LIGNAN LEVELS IN MONOCOT SEEDS/STRATEGIES TO INCREASE LIGNAN PRODUCTION

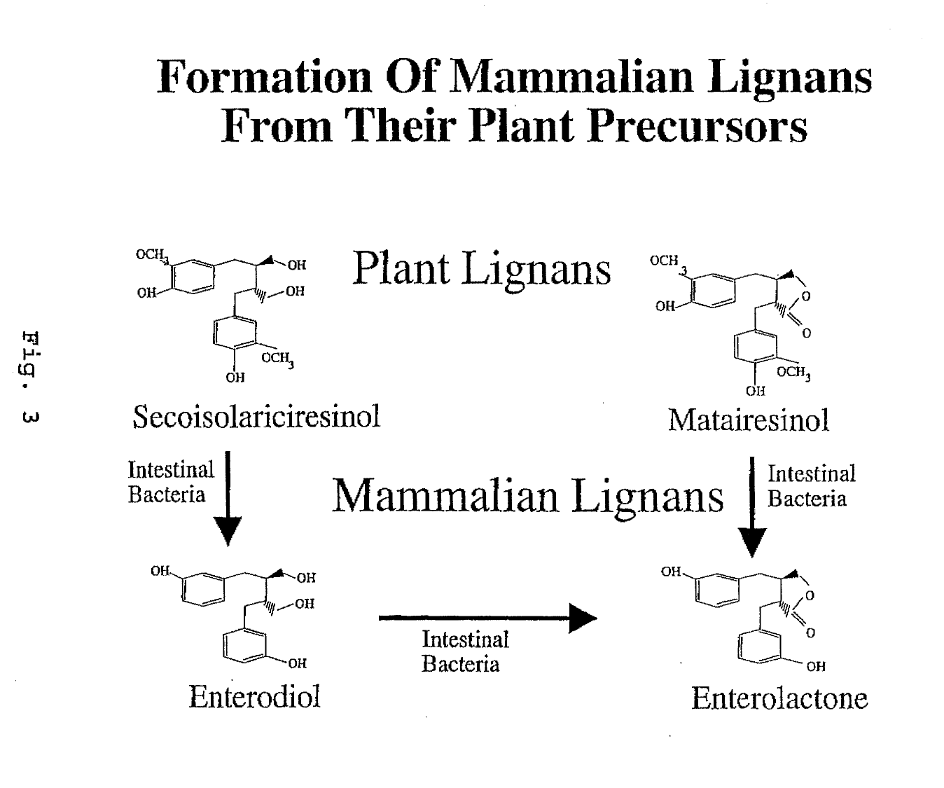

As indicated in FIG. 3 human lignans are derived from plant lignans through the action of colonic bacteria. The plant lignan, secoisolariciresinol, is converted to enterodiol and then to enterolactone while matairesinol is converted to enterolactone directly. Recently, the pathway for matairesinol production in Forsythia intermedia has been elucidated (FIG. 2) and a number of genes which encode enzymes responsible for lignan biosynthesis in the phenylpropanoid pathway have been cloned, laying the foundation for the metabolic-engineering of lignans in crop plants. (See e.g., Davin et al., 2000.)

The present invention is applicable to the generation of monocots exhibiting modified levels of G-lignans in seed tissues in which they are typically found. The specific seed tissue and/or maturation state in which G-lignans are typically found may vary between plant species. Using the methods of the present invention, a G-lignan, e.g., matairesinol, is produced in seeds of transformed plants in an amount greater than the amount found in seeds of untransformed plants. In other words, the concentration of any one of the above-described G-lignans in transformed seeds exceeds the amount found in untransformed seeds, when assayed by a conventional assay system for detecting lignan(s), for example, gas chromatography-mass spectroscopy (GC-MS; GC-MS-SIM) or high performance liquid chromatography (HPLC).

In practicing the method, chimeric genes or heterologous nucleic acid constructs containing the coding sequence for one or more proteins or enzymes integral to the phenylpropanoid biosynthetic pathway for production of G-lignans are introduced into a target plant to alter production of G-lignans. In making such alterations, it is generally preferred that the gene target, promoter and tissue be endogenous to the plant of interest, and that expression occur in a tissue and developmental stage consistent with that normally found in nature. Therefore, preferred strategies for increasing lignan content in monocot seeds take advantage of existing biosynthetic pathways utilized by the native (wild-type) plant, that are seed tissue and/or developmental stage specific. Preferred protein and enzyme targets for inclusion in chimeric gene constructs used to produce the transgenic monocot plants and seeds of the present invention are those which facilitate rate limiting steps in the phenylpropanoid pathway leading to G-lignan formation, examples of which include dirigent protein; laccase; pinoresinol/lariciresinol reductase (reductase) and secosisolariciresinol dehydrogenase (dehydrogenase).

Exemplary monocots for introduction of the chimeric gene constructs of the present invention include, but are not limited to, wheat, maize, barley, rice, millet, oats, rye, triticale, sorghum, and corn.

The steps involved in modification of lignan levels in monocot seeds include, but are not limited to, the following; (1) determining in which seed tissue G-lignans are found by analysis of G-lignans, e.g. lariciresinol and secoisolariciresinol diglucoside, in various plant tissues and at various stages of development for the target plant species, followed by; (2) selecting an appropriate promoter based on the target seed tissue and seed developmental stage; and (3) assembling a chimeric gene construct including the promoter, the target gene and appropriate regulatory elements necessary for expression in seeds of a monocot plant.

Candidate target genes include those which encode proteins integral to the biosynthetic pathway leading to G-lignan formation, as described above, and will be found in the seed tissue in which G-lignans are normally found. Exemplary target enzymes for modification include, but are not limited to dirigent protein; laccase; pinoresinol/lariciresinol reductase; and secosisolariciresinol dehydrogenase.

A typical chimeric gene of the present invention, for transformation into a plant, may include a transcriptional regulatory region inducible during seed development (e.g., a seed tissue specific promoter such as a seed-induced promoters include the barley β-amylase (Kreis, et al., 1987) and β-glucanase gene promoters (Wolf, 1992), or a seed developmental stage specific promoter, such as a promoter for the rice glutelin multigene family, Gt1, Gt2, Gt3, GluA-3, and GluB-1, described, for example, in Takaiwa, et al., 1991a, 1991b; Okita, et al., 1989; Abe et al., 1989; Kim et al., 1990. Such a typical chimeric gene will also include a protein coding sequence, and a termin.ator sequence.

Preferred promoters for use in the present invention include, for example, known tissue specific promoters for the seed tissue and/or developmental stage in which G-lignans are normally found, preferably promoters derived from the same plant species as the plant in which the chimeric gene construct will be introduced, and native (wild type) promoters for the gene encoding the target protein.

Expression of native (wild type) genes for the various enzymes and proteins of the phenylpropanoid biosynthetic pathway may yield varying levels of transcript depending upon the tissue type and developmental stage of the plant in which they are expressed.

The methods of the invention include stably transformin.g a monocot plant with a chimeric gene having (1) an appropriate promoter, as described above, (2) a first DNA sequence encoding a protein integral to the lignan biosynthetic pathway, (3) control elements necessary for expression of the protein coding sequence, and may further include (4) a second DNA sequence encoding a signal polypeptide. In such cases, the second DNA sequence is operably linked to the transcriptional regulatory region and the first DNA sequence, and the signal polypeptide are in translation-frame with the protein coding sequence. Expression of form of the protein having a signal sequence is effective to facilitate secretion of the protein across the aleurone or scutellar epithelium layer of seeds into the endosperm.

IV. METHODS AND COMPOSITIONS OF THE INVENTION

The phenylpropanoid pathway that leads to lignan formation has three branches at the point of formation of para-coumaryl alcohol, coniferyl alcohol and sinapyl alcohol, leading to “H”, “G” and “S” lignans, respectively. Using the methods of the present invention, it is possible to introduce a chimeric gene construct containing a DNA sequence encoding a protein integral to the phenylpropanoid pathway leading to G-lignan formation into monocot plants, and increase the G-lignan content in seeds, or within certain seed tissues, such as the seed testa layer, pericarp, aleurone and endosperm, without significantly interfering with plant growth and development, or phenotypic characteristics of the plant or seed.

The present invention provides transformed monocot plants and methods of making them wherein the plants produce higher levels of G-lignan precursors or G-lignans, at levels and in specific tissues that allow the plant to develop normally and without interfering with seed production levels. Chimeric genes including DNA sequences encoding one or more proteins integral to the phenylpropanoid pathway leading to G-lignan formation, plant expression vectors, monocot plant cells bearing such chimeric genes or transformed by such expression vectors, transformed monocot plants and seeds having elevated levels of G-lignans (e.g., secoiso-lariciresinol, matairesinol), are thus provided. Seeds from such transformed monocot plants may upon mammalian consumption, provide associated positive health benefits, as further detailed below.

More specifically, one aspect of the present invention is directed to a method of increasing G-lignan content in monocot seeds by up-regulating in developing, maturing, or germin.ating seeds, one or more of the following proteins or enzymes: dirigent protein; laccase; pinoresinol/lariciresinol reductase (reductase); and secosisolariciresinol dehydrogenase (dehydrogenase), all of which are integral to the branch of the phenylpropanoid pathway leading to formation of monolignols or dimeric lignan precursors and G-lignans. Specifically, the production of one or more of the above proteins is up-regulated by introducing into a monocot plant or plant cell, a chimeric gene having (i) a seed tissue or seed developmental stage specific transcriptional regulatory region induced during seed development, maturation or germin.ation (e.g., a seed specific promoter), and (ii) a gene encoding a protein integral to the lignan biosynthetic pathway operably linked to the transcriptional regulatory region.

In an alternative approach, the production of one or more of the above proteins may be up-regulated by introducing into a monocot plant or plant cell, a chimeric gene having (i) the endogenous promoter for the target protein, operably linked to (ii) multiple copies of a gene encoding one of the above-described proteins. The protein is preferably expressed in plant tissues which normally produce lignans by using a transcription regulatory region specific for various seed tissues, such as the seed testa layer, pericarp, aleurone and endosperm.

In a preferred approach, the coding sequence for multiple proteins integral to the lignan biosynthetic pathway are expressed in the same plant cell at the same time, e.g., the coding sequence for dirigent protein; laccase; pinoresinol/lariciresinol reductase (reductase); and secosisolariciresinol dehydrogenase (dehydrogenase).

In a related aspect, the invention includes a monocotyledonous plant capable of producing seeds with elevated G-lignan content, and monocot seeds produced by the plant.

Also disclosed is a seed composition for use in stereospecifically converting E-coniferyl alcohol to G-lignans, e.g. (+)-pinoresinol, (+)-lariciresinol, (−)-secoisolariciresinol, (−)-secoisolariciresinol diglucoside, and in particular, to (−)-matairesinol (see FIG. 2). The seed composition contains seeds from a monocot plant which has been stably transformed and contain a gene encoding one or more of the following proteins: a dirigent protein together with laccase capable of converting E-coniferyl alcohol stereospecifically to a selected enantiomer of pinoresinol; pinoresinol lariciresinol reductase; and secosisolariciresinol dehydrogenase, operably linked to a seed-specific promoter or their respective endogenous promoters. Due to selective expression of the transgenes in seed tissues, the resulting seed composition is effective to produce significant amounts of one or more of the above-described optically active G-lignans, which are derived from coniferyl-alcohol.

In a related aspect, a method of formin.g a lignan-enriched seed composition is disclosed.

In one alternative approach, over expression of selected gene products integral the G-lignan biosynthetic pathway is carried out together with inhibition of the expression of proteins which facilitate formation of metabolites other than G-lignans in order to produce elevated levels of G-lignans in the seeds of transgenic monocot plants.

In another alternative approach, the expression of selected gene products integral to the front end of the phenylpropanoid biosynthetic pathway is up-regulated by introduction and stable expression of chimeric gene constructs as a means to increase G-lignan levels in monocot seeds including, but not limited to, one or more of (1) phenylalanine ammonia lyase (PAL); (2) cinnamate-4-hydroxylase (C4H); and (3) caffeate-3-hydroxylase (C3H).

In a further alternative approach, the expression of selected gene products integral to the front end of the phenylpropanoid biosynthetic pathway is downregulated, including, but not limited to: (1) ferulate-5-hydroxylase (F5H); and (2) chalcone synthase (CS).

Following introduction of a chimeric gene construct containing the coding sequence for a gene product integral to the G-lignan biosynthetic pathway, plant tissue may be analyzed for proteins and nucleic acids indicative of modified G-lignan expression, as further described below.

C. CHIMERIC GENE CONSTRUCTS

In order to increase the absolute or relative concentrations of G-lignans in plant seeds, the expression of various intermediates integral to the G-lignan biosynthetic pathway are modified by growing a plant transformed with an expression vector containing a chimeric gene construct under the appropriate conditions to induce or cause expression of the protein (e.g. dirigent protein or an enzyme). The conditions appropriate, for example, for enzyme expression will vary with the choice of the expression vector, the target plant tissue and developmental stage, and will be easily ascertained by one skilled in the art through routine experimentation. For example, the use of constitutive promoters in the expression vector will require optimizing the growth and proliferation of the plant cell, while the use of an inducible promoter requires the appropriate growth conditions for induction. For example, if the promoter is developmental stage specific (e.g. inducible during seed development), the appropriate environmental stimulus will be required for the promoter to be induced and the chimeric gene construct to be expressed, e.g., sugar depletion in culture or water uptake followed by gibberellic acid production in germin.ating seeds.

Numerous strategies may be pursued to increase lignan production in seeds, including, but not limited to, the following.

Expression vectors for use in this aspect of the invention comprise a chimeric gene construct (or expression cassette), designed for operation in plants, particularly monocot plants, with companion sequences upstream and downstream from the expression cassette. The companion sequences will be of plasmid or viral origin and provide necessary characteristics to the vector to permit the vectors to move DNA from bacteria to the desired plant host.

A monocot plant may be stably transformed with a chimeric gene having (i) a promoter, for example, a native promoter for the target gene; a seed tissue specific promoter; or a seed-developmental stage specific promoter induced during seed development, maturation or germin.ation, and (ii) a gene sequence whose expression ultimately results in production of G-lignans, or whose expression is effective to block formation of a metabolite of a competing biosynthetic pathway or branch point which does not lead to formation of G-lignans.

The expression construct also utilizes additional regulatory DNA sequences e.g., preferred codons, termin.ation sequences, to promote efficient translation of the gene encoding the protein, as will be described.

In addition to encoding the protein of interest, the chimeric gene expression cassette may encode a signal peptide that allows processing and translocation of the protein, as appropriate. Preferred signal sequences are those corresponding to the RAmy3D rice promoter and Ramy1A promoter, respectfully. A plant signal sequence is placed in frame with a heterologous nucleic acid encoding a peptide or protein such that signal peptidase cleavage occurs precisely at the start of the mature peptide.

The expression cassette or chimeric gene(s) in the transformation vector typically have a transcriptional termin.ation region at the opposite end from the transcription initiation regulatory region (promoter), essentially as defined above. The transcriptional termin.ation region may normally be associated with the transcriptional initiation region or from a different gene. The transcriptional termin.ation region may be selected, particularly for stability of the mRNA to enhance expression. Illustrative transcriptional termin.ation regions include the NOS termin.ator from Agrobacterium Ti plasmid and the rice β-amylase termin.ator.

Polyadenylation tails, (Alber et al., 1982) are also commonly added to the expression cassette to optimize high levels of transcription and proper transcription termin.ation, respectively. Polyadenylation sequences include, but are not limited to, the Agrobacterium octopine synthetase signal, Gielen et al,, 1984, or the nopaline synthase of the same species, Depicker et al., 1982.

Since the ultimate expression of the chimeric gene construct will be in a eukaryotic cell (in this case, a member of the grass family), it is desirable to determin.e whether any portion of the cloned gene contains sequences which will be processed out as introns by the host's splicing machinery. If so, site-directed mutagenesis of the “intron” region may be conducted to prevent losing a portion of the genetic message as a false intron code (Reed et al., 1985).

Chimeric gene constructs containing a gene encoding a protein integral to the phenylpropanoid biosynthetic pathway may also include a selectable marker for use in plant cells. Such selectable markers may be either on the same plasmid or in the form of separate plasmids, including, but not limited to, the nptII kanamycin resistance gene, for selection in kanamycin-containing media, or the phosphinothricin acetyltransferase gene, for selection in medium containing phosphinothricin (PPT).

The vectors may also include sequences that allow their selection and propagation in a secondary host, such as, sequences containing an origin of replication and a selectable marker such as antibiotic or herbicide resistance genes. Typical secondary hosts include bacteria and yeast. In a representative approach for formin.g an expression vector for use in the present invention, the secondary host is Escherichia coli, the origin of replication is a colE1-type, and the selectable marker is a gene encoding ampicillin resistance. Such sequences are well known in the art and are commercially available as well (e.g., Clontech, Palo Alto, Calif.; Stratagene, La. Jolla, Calif.).

A vector for use in the present invention may also be modified to form an intermediate plant transformation plasmid that contains a region of homology to an Agrobacterium tumefaciens vector, a T-DNA border region from Agrobacterium tumefaciens, and chimeric genes or expression cassettes (described above). Further, the vector may comprise a disarmed plant tumor inducing plasmid of Agrobacterium tumefaciens.

The appropriate nucleic acid sequence may be inserted into the vector by a variety of procedures known to those of skill in the art. In general, DNA is inserted into an appropriate restriction endonuclease site(s) using techniques known in the art. Construction of suitable vectors containing the components set forth above employs standard ligation techniques which are known to the skilled artisan.

The construction of such expression vectors is generally known in the art. Specific examples provided here include expression vectors comprising the coding sequence for laccase, dirigent protein, lariciresinol reductase and secoisolariciresinol dehydrogenase, as further described in Example 2.

B. PROMOTERS

Transcriptional regulatory or promoter sequences for use in the methods described herein may be either constitutive or inducible promoters. The promoters may be either naturally occurring promoters or hybrid promoters. Hybrid promoters, which combine elements of more than one promoter, are known in the art, and may be used in practicing the present invention.

B 1. KNOWN PROMOTERS

Promoters for regulating expression of any one of the herein described genes are preferably seed-specific promoters, that is, promoters which induce transcription of a downstream coding sequence preferentially in seeds, relative to other types of plant tissues. Moreover, the seed-specific promoter may be seed-tissue specific, i.e., induced preferentially in a particular seed tissue such as the seed testa layer, pericarp, aleurone or endosperm, and/or seed developmental stage specific, i.e., induced in seeds during a certain stage of seed development, in response to either endogenous factors present in the seed, plant hormones, such as abscissic or gibberellic acid, or physical stimuli, e.g., heat or moisture, or the presence or absence of a small molecule, such as the reduction or depletion of sugar, such as sucrose, in culture medium, or in plant tissues, such as germin.ating seeds. In this way, chimeric gene constructs affecting plant regulatory and developmental pathways are expressed preferentially and preferably solely in seeds of the plant, thereby allowing the plant to develop without significantly interfering with the phenylpropanoid pathway in other plant tissues and organs. Seed-specific expression is also desirable since the naturally-occurring plant lignans, secoisolariciresinol diglucoside and matairesinol, are found predomin.antly in the outer seed layers of representative cereals such as rye (Nilsson et al., 1997). Thus, the patterns of lignan production in transformed plants will be designed to closely follow those of wild-type plants, to produce as little phenotypic variation as possible (outside of enhanced G-lignan biosynthesis) in transformed monocot seeds relative to native seeds.

Promoters for use in the present invention are preferably derived from cereals such as rice, barley, wheat, oat, rye, millet, triticale, sorghum, and corn.

Alternatively, a seed-specific promoter from a non-cereal monocot may be used, and modified according to techniques well known in the art for seed-specific expression of the enzyme-coding sequences described herein. Exemplary promoters are described below.

In determining how the chimeric gene constructs encoding proteins integral to the phenylpropanoid pathway will be expressed, relevant factors include the type of promoter, the temporal pattern of the promoter (e.g. with respect to the developmental stage of the plant or the plant tissue), and the operation of the promoter based on its position within the genome. A promoter which is expressed concurrently with or prior to the normal activation of the native gene sequence is preferred. Alternatively, the protein may be made at a significantly higher concentration than is normally seen, through the use of a inducible promoter or high expression promoter, such that the protein is made at increased concentration levels.

B2. SEED TISSUE SPECIFIC

The present invention takes advantage of promoters specific to the various seed tissues, including, but not limited to the pericarp, testa layer, aleurone and the endosperm. Such seed tissue specific promoters may also be expressed at varying levels depending upon the developmental stage of the seed (Okita et al., 1989).

Promoters from seed tissue specific genes such as those described in Muller and Knudsen, 1993, the contents of which is expressly incorporated herein by reference, as well as the references contained therein, are suitable for use in the chimeric gene constructs of the present invention.

Representative seed tissue specific promoters include promoters that direct endosperm-specific expression (Takaiwa et al., 1991a, 1991b; Okita et al., 1989; Abe et al., 1989; Kim et al, 1990).

During germin.ation of the barley grain, two μ-glucanase isozymes (E.C.3.2.1.73) are synthesized in the aleurone (isozyme II) and in the aleurone and scutellum (isozyme II), then secreted into the endosperm (Wolf, 1992).

Additional seed-induced promoters include the barley μ-amylase promoter (Kreis et al., 1987) and promoters from the barley hordein gene family, e.g., B-, C-, and D-hordein genes (Sorensen et al., 1996 and references therein; Sorensen, 1992; Brandt et al., 1985; Entwistle et al., 1991; Muller et al., 1993). B- and C-, hordein polypeptides are major storage proteins synthesized in the endosperm (Brandt et al., 1985), and hordein gene promoters appear to direct the specific expression of the corresponding genes in the endosperm. A 12S seed storage promoter, such as that isolated from oat (Avena sativa) may be used in the chimeric gene constructs of the present invention to direct endosperm specific expression (Schubert et al., 1990).

Additional seed-induced promoters which direct production of seed storage products for use in the invention are the maize zein gene promoter (Bianchi et al., 1988), and promoters from wheat glutenin genes, such as those described in Anderson et al., 1989 and Halford et al., 1989. The endosperm-specific expression regulated by both the barley B-hordein promoter and the maize zein promoter has been correlated with hypomethylation of the corresponding structural genes (Sorensen et al., 1996; Bianchi et al., 1988).

Also useful in the present invention are seed induced corn promoters. Promoters from cloned seed genes expressed in specific tissues in corn include: the corn O2-opaque 2 gene promoter, which regulates expression of a zein polypeptide expressed in the endosperm (Schmidt, et al., 1987); the corn Sh2-shrunken 2 gene promoter (Shaw et al., 1992) and the Bt2-brittle 2 gene promoter (Bae et al., 1990), which together regulate starch synthesis in the endosperm; the Zp1 zein gene promoter (Burr et al., 1982); the zmZ27 zein gene promoter; a rice small subunit ADP-glucose phosphorylase gene promoter, osAGP; and the rice glutelin 1 gene promoter, all of which direct endosperm specific expression, as well as the promoter for maize granule-bound starch synthase (Waxy) gene, zmGBS, which directs expression in the pollen and the endosperm (Russell et al., 1997).

Also for use in the present invention are the Agp1 and Agp2 gene promoters (Giroux, M. J., Hannah, L. C., 1994), the corresponding genes for which are expressed in both the embryo and endosperm.

Additional seed-induced promoters include the barley B22E gene promoter, which is differentially expressed in immature aleurone layers, but has been shown to direct expression in both the aleurone and pericarp cells of immature barley grains (Klemsdal et al., 1991).

Other suitable promoters include the sucrose synthase and sucrose-6-phosphate-synthetase (SPS) promoters from rice and barley. A promoter directing expression of selectable markers used for plant transformation (e.g., nptII) should operate effectively in plant hosts. One such promoter is the nos promoter from native Ti Plasmids (Herrera-Estrella et al., 1983). Others include the 35S and 19S promoters of cauliflower mosaic virus (Odell et al., 1985), and the 2′ promoter (Velten et al., 1984).

B3. SEED DEVELOPMENTAL STAGE SPECIFIC

Representative seed developmental stage specific promoters include the promoters from the rice glutelin multigene family, Gt1, Gt2, Gt3, GluA-3, and GluB-1, which are active during seed development and direct endosperm-specific expression (Okita, et al., 1989; Kim et al., 1990). Promoters from the rice glutelin multigene family were first detected 6 days after flowering and reached a maximum at 14 days after flowering, then subsequently declined (Takaiwa et al., 1991b). Various rice seed storage proteins (albumin., prolamin., and type II glutelin) were shown to be expressed over a short time window after flowering (i.e. maximum detection at 15 days after flowering) suggesting that they share transcriptional machinery (Nakase et al., 1996).

Exemplary promoters for directing expression in germin.ating seeds include the rice β-amylase RAmy1A promoter, which is up-regulated by gibberellic acid; and the rice β-amylase RAmy3D and RAmy3E promoters which are strongly up-regulated by sugar depletion in the culture. These promoters are also active during seed germin.ation. Representative promoters further include the promoters from the rice β-amylase RAmy1A, RAmy1B, RAmy2A, RAmy3A, RAmy3B, RAmy3C, RAmy3D, and RAmy3E genes, and from the pM/C, gKAmy141, gKAmy155, Amy32b, and HV18 barley β-amylase genes. These promoters are described, for example, in ADVANCES IN PLANT BIOTECHNOLOGY Ryu, D. D. Y., et al, Eds., Elsevier, Amsterdam, 1994, p.37, and references cited therein.

Additional seed-induced promoters include the barley B22E gene promoter, which is differentially expressed in immature aleurone layers, but has been shown to direct expression in both the aleurone and pericarp cells of immature barley grains (Klemsdal, et al., 1991).

Exemplar preferred promoters include the rice glutelin Gt1 promoter and the aleurone-specific chitinase gene promoter (Chi26).

B4. PROMOTER ISOLATION

In addition, promoters for use in the methods of the invention may be isolated from various tissues and/or at various stages of seed development by a variety of techniques routinely used by those of skill in the art. Target tissues for isolation of such promoters include, but are not limited to, the seed testa layer, pericarp, aleurone and the endosperm.

The testa, or seed coat, is an outer layer of cells adjacent to the aleurone layer surrounding the endosperm. The testa is composed of an inner and outer cuticle, often impregnated with waxes and fats, and one or more layers of thick-walled protective cells. The testa may contain layers of crystal containing cells composed of calcium oxalate or carbonate; the seed coat may also contain mucilaginous cells that burst upon contact with water, to provide a water-retaining barrier. The testa layer in seeds functions to provide a protective barrier between the embryo and the external environment.

The pericarp, is the outer covering of a grain or seed. The aleurone layer surrounds the endosperm and is a living tissue which does not store many reserves, but may be responsible for the release and mobilization of enzymes, such as proteinases. (Bewley, J D and Black, M., Eds., Seeds Physiology of Development and Germin.ation, page 324, Plenum Press, New York, 1994). The endosperm is the major storage reservoir for carbohydrate reserves in cereals.

Various strategies for promoter isolation from RNA extracts of each target tissue at the appropriate developmental stage are employed to isolate a tissue specific promoter which may then be incorporated into a chimeric gene construct along with a gene encoding a protein which is integral to the biosynthetic pathway leading to G-lignan formation.

Promoters may also be obtained from an alternative monocot species. For example, a suitable promoter such as the Gt1 gene promoter from rice may be isolated from nucleic acid containing extracts derived from other cereals, e.g., wheat, oat, or the like, using conventional hybridization techniques routinely used by those of skill in the art.

Such techniques include, but are not limited to; (1) use of conventional hybridization techniques and known promoters from a different species, tissue and/or developmental stage to obtain related sequences from the target monocot species, seed tissue or seed developmental stage, (2) subtractive hybridization (Lee et al., 1991), (3) differential display (Liang et al., 1992; Bauer et al., 1993), and (4) selective amplification via biotin-and restriction-mediated enrichment (SABRE; Lavery et al., 1997).

The present invention takes advantage of a seed tissue specific or developmental stage specific promoter, i.e., a promoter that is selectively induced in a particular seed tissue or temporally expressed at various times during seed development.

Conventional hybridization techniques are applied to identifying the sequence of known promoters sequences from monocots other than the one in which they have been found. By way of example, to identify a wheat endosperm-specific promoter, a cDNA library from wheat seeds, or wheat endosperm, is amplified using probes containing consensus sequences from, e.g.. the known corn, promoter sequence. The sequence and location of the promoter sequence for the corn zein gene, Sh2 (Shaw et al., 1992) may be used to identify related promoter sequences in wheat or other monocots. The identified cDNA is then sequenced, and may be incorporated into a chimeric gene construct with the appropriate target gene, according to the methods described herein.

Sequences present in one nucleic acid sample and not another may also be derived by subtractive hybridization where nucleic acid extracts are prepared from two samples, hybridized and sequences that do not hybridize are amplified (Lee et al., 1991).

Alternatively, cDNA difference techniques (e.g., a form of representational difference analysis for cDNA as described in Hubank et al., 1994) can be employed to identify a gene that is expressed in one type of seed tissue, e.g. in the endosperm, but not in other seed cells, such as aleurone cells.

In another approach for identifying cDNAs for testa-specific transcripts, a differential display technique is utilized to systematically identify, by their cDNAs, the messenger RNAs expressed in one type of tissue sample and not in another. (Liang et al., 1992; Bauer et al., 1993).

The Bauer et al. variation of this technique, called differential display reverse transcription polymerase chain reaction (DDRT-PCR) has been applied to differential display of mRNAs expressed at different stages of liver regeneration. The modified techniques has the added features of a specific PCR primer design, running sequencing gels under non-denaturing conditions and automatic recording of the cDNA pattern, allowing for more efficient analysis of sequence differences between 2 mRNA samples.

In an additional approach called selective amplification via biotin- and restriction-mediated enrichment (SABRE; Lavery et al., Genetics 94:6831-6836, 1997), species more abundant in one double-stranded DNA population (the tester) than in another (the driver) can be enriched relative to species equally expressed in both by denaturing a mixture of both populations and allowing it to reassociate, and then isolating only double-stranded molecules of which both strands are derived from the tester population (tester homobybrids).

Once a tissue-specific or developmental stage specific gene is isolated, for example by the difference techniques discussed above, the full gene may then be sequenced and the promoter region is identified. Identifying the promoter region is performed by standard analysis, for example, by identifying a start codon at the beginning of an open reading frame, and promoter-specific sequences, e.g., a TATA box and transcription initiation site upstream of the start codon. The promoter can then be isolated and cloned by standard recombinant methods, for use in the chimeric gene of the invention.

Following isolation of the promoter, expression specificity can be further confirmed by fusing the promoter in-frame to a reporter such as the synthetic gene of the jelly fish green fluorescence protein, engineered for high level expression in higher plants (Chiu, et al., 1996). The promoter-reporter gene construct is then delivered on gold particles by the biolistic method into the target tissue(s) and control tissues (e.g., leaf, stem), as described below. Expression specificity can also be confirmed by Northern blot analysis using mRNA preparations from a variety of barley tissues.

B5. ANTISENSE STRATEGY

As a general approach, an inhibitory transcript RNA produces its biological effect by inhibiting expression of an enzyme or protein that is a component integral to a branch point of the phenylpropanoid pathway leading to formation of metabolites other than G-lignans.

The sense strand of the inhibitory gene sequence may be complementary to the transcribed region of the sense strand of the target gene. That is, the orientation of the inhibitory gene sequence in the chimeric gene is in the reverse direction (3′-5′), so that RNA transcribed from the plasmid is complementary in sequence to the mRNA transcribed from the corresponding endogenous gene (i.e. antisense).

Antisense inhibition with an antisense nucleic acid sequence can take place in two independent ways: the antisense sequence can control the transcript level in the nucleus and/or the translation efficiency of the target mRNA in the cytoplasm (Cornelissen et al., 1989; Cornelissen, 1989). It has been demonstrate that introduction of either sense or anti-sense constructs for an O-methyl transferase (OMT) can result in altered lignin composition in transgenic tobacco plants (Atanassova et al., 1995).

A monocot plant may be genetically altered by transformin.g the plant with a chimeric gene having (i) a transcriptional regulatory region (e.g., a promoter) expressed in a specific seed tissue, or induced during seed development, (ii) an inhibitory gene sequence operably linked to the transcriptional regulatory region for induction of an inhibitory transcript RNA, and (iii) additional regulatory DNA sequences and/or a sequence encoding a signal peptide, as appropriate to promote efficient expression of the inhibitory sequence.

Such an inhibitory nucleic acid sequence is capable of inhibiting expression of a gene encoding a protein or enzyme that normally is at a branch point in the phenylpropanoid pathway that leads to formation of metabolites other than G-lignans. By way of example, enzymes that occur at such branch points include, but are not limited to, ferulate-5-hydroxylase (F5H) which catalyzes the conversion of ferulate to 5-hydroxyferulate and chalcone synthase (CHS) which catalyzes formation of flavenoid compounds composed of a basic 15-carbon flavenoid skeleton including e.g., anthocyanins, glycosylated flavenoid pigments, starting with para-coumaryl CoA. Buildup of ferulate resulting from a lack of enzymatically active F5H or defective F5H will favor the pathways leading to H and G-lignan formation by blocking the pathway leading to S-lignan formation.

Inclusion of such an inhibitory nucleic acid sequence in a chimeric gene construct of the invention is effective to prevent (i) transcription of the gene encoding e.g. F5H or CHS; or (ii) translation of the F5H or CHS mRNA. Preferably, the inhibitory sequence is an antisense F5H or CHS sequence effective to allow an accumulation of ferulate or para-coumaryl CoA within transformed cells. Preferably, the inhibitory sequence is placed under the control of a seed tissue specific or seed developmental stage specific promoter. In one aspect, the promoter is a stage specific promoter that is activated in seed at the appropriate developmental stage of the seed.

It will be appreciated that the inhibitory transcript RNA may also be designed to block transcription by binding to one strand of the nucleic acid during transcription. The transcript in this case may have either sense or antisense orientation with respect to the target gene sequence. Further, the transcript can target any sequence of the gene, including transcription binding site(s) and intron regions, effective to block transcription of a full-length mRNA. Such non-cDNA sequences can be identified by isolation and sequencing of the chalcone synthase (CHS) gene from a genomic library, according to standard procedures.

For example, constitutive expression of an antisense chalcone synthase gene in transgenic petunia and tobacco plants has been shown to reduce levels of both mRNA for the enzyme and the enzyme itself (Van der Krol et al., Nature 333:866-869, 1988)

The structure of the chalcone synthase (GenBank Accession No. X92548) gene which encodes the CHS enzyme or protein in rye is presented herein by way of example. It will be understood that nucleotide differences in the CHS gene may exist among different genotypes and among various monocots, and that the invention is intended to encompass such variants, as defined above. It will be further understood that sequences as short as 10-20 bases, can be used for antisense inhibition.

Construction of a chimeric gene construct containing an inhibitory sequence, e.g., an antisense nucleic acid sequence, may be accomplished as described in van der Krol et al., 1988.

An exemplary procedure for preparing an antisense construct of the present invention includes obtaining a cDNA fragment from a known enzyme in a branch point in the phenylpropanoid biosynthetic pathway, e.g., a chalcone synthase (CHS) gene sequence. That sequence may be used to determin.e the complementary sequence by preparing a lambda cDNA library as described in Kristiansen and Rohde (1991) and screening with DNA derived from the target monocot species. Positive clones are identified, and a cDNA insert about 20 nucleotides to 1.3 kb in length, corresponding to a portion of the gene sequence, may then be subcloned into a subcloning vector such as pUC9.

A clone containing the complementary sequence may then be partially digested with a restriction enzyme, e.g., NcoI, and EcoRI, and the sticky ends made blunt using the Klenow fragment of DNA polymerase I and dNTPs. Various resulting blunt ended fragments are then subcloned, e.g., into the HindII site of phage M13 mp7, to allow isolation of a BamHI fragment. This fragment can then be cloned into, e.g., the BamHI site separating the promoter and a nopaline synthase 3′ flanking region containing a polyA tail, and inserted into the polylinker site of a suitable plant transformation vector, to form an antisense construct.

An antisense chimeric gene construct as described above, may be introduced into monocot plant cells by the methods described below resulting in production of transgenic plants and seeds wherein the level of a target protein or enzyme is reduced or elimin.ated.

V. PRODUCING TRANSGENIC PLANTS

A. METHODS OF DNA INTRODUCTION

Following introduction of a chimeric gene construct encoding a protein integral to the biosynthetic pathway leading to G-lignan formation or an antisense chimeric gene construct encoding a protein which facilitates formation of metabolites other than G-lignans, the coding sequence contained within the chimeric gene construct is expressed, either in cell culture, or in germin.ating seeds.

The plants to be transformed are monocot plants, particularly the members of the taxonomic family known as the Gramin.eae. This family includes all members of the grass family of which the edible varieties are known as cereals. The cereals include a wide variety of species such as wheat (Triticum sps.), rice (Oryza sps.) barley (Hordeum sps.) oats, (Avena sps.) rye (Secale sps.), corn (Zea sps.) and millet (Pennisettum sps.). In the present invention, preferred family members are rice, corn, wheat and barley.

The transformation of plants may be carried out in any of the various ways known to those skilled in the art of plant molecular biology. See, Wu and Grossman (1987) incorporated herein by reference. Such techniques include, but are not limited to, e.g., electroporation, protoplast fusion and microparticle bombardment. Various methods for direct or vectored transformation of plant cells, such as plant protoplast cells, have been described, e.g., in PCT application WO 95/14099.

By transformation is meant alteration of the genotype of a host plant by the introduction of a nucleic acid sequence, e.g., a chimeric gene construct or heterologous nucleic acid construct of the type described herein. The nucleic acid sequence need not necessarily originate from a different source, but it will, at some point, have been external to the cell into which it is to be introduced.

In one approach, the nucleic acid is mechanically transferred by microinjection directly into plant cells by use of micropipettes. Alternatively, the foreign nucleic acid may be transferred into the plant cell by using polyethylene glycol, which forms a precipitation complex with the genetic material that is taken up by the cell (Paszkowski, et al., 1984).

A chimeric gene construct may also be introduced into the plant cells by electroporation (Fromrnm, et al., 1985). Electrical impulses of high field strength reversibly permeabilize biomembranes, allowing the introduction of Plasmids or nucleic acids containing the chimeric gene constructs into plant protoplasts. Electroporated plant protoplasts reform the cell wall, divide, and form a plant callus. Selection of the transformed plant cells with the transformed gene can be accomplished using phenotypic markers.

Transformation of monocot plants is preferably carried out as generally described in Jensen, et al., 1996, and in Wan, et al., 1994. In this procedure, the chimeric gene construct-plasmid is adsorbed to gold particles (e.g., 1.0 μm particles, DuPont, Wilmin.gton, Del.) and delivered into immature embryos by particle bombardment. When two or more plasmid DNA constructs are used to transform plant tissue, a mixture of the two or more plasmids may be adsorbed to the particles for co-injection.

Preferred methods of transforming wheat and sorghum are generally described in Nehra et al., 1994.

Particle bombardment may be used alone or in combination with other methods such as Agrobacterium-mediated transformation. In a typical transformation methodology, the embryo and endosperm of mature seeds are removed to expose scutulum tissue cells. The cells may be transformed by DNA bombardment or injection, or by vectored transformation, e.g., by Agrobacterium infection after bombarding the scuteller cells with microparticles to make them susceptible to Agrobacterium infection (Bidney et al., 1992).

Another method of introducing the nucleic acid constructs of the invention into plant cells is to infect a plant cell, an explant, a meristem or a seed with Agrobacterium tumefaciens transformed with the segment. Under appropriate conditions known in the art, the transformed plant cells are grown to form shoots, roots, and develop further into plants. The nucleic acid segments can be introduced into appropriate plant cells, for example, by means of the Ti plasmid of Agrobacterium tumefaciens. The Ti plasmid is transmitted to plant cells upon infection by Agrobacterium tumefaciens, and is stably integrated into the plant genome and preferred methods for Agrobacterium-mediated transformation of corn and/or rice are generally disclosed in Horsch et al., 1984 and Fraley et al., 1983.

Ti Plasmids contain two regions essential for the production of transformed cells. One of these, named transfer DNA (T DNA), induces tumor formation. The other, termed virulent region, is essential for the introduction of the T DNA into plants. The transfer DNA region, which transfers to the plant genome, can be increased in size by the insertion of the foreign nucleic acid sequence without its transferring ability being affected. By removing the tumor-causing genes so that they no longer interfere, the modified Ti plasmid can then be used as a vector for the transfer of the gene constructs of the invention into an appropriate plant cell, such being a “disabled Ti vector”. All plant cells which can be transformed by Agrobacterium and whole plants regenerated from the transformed cells can also be transformed according to the invention so as to produce transformed whole plants which contain the transferred target nucleic acid sequence.

There are presently at least three different ways to transform plant cells with Agrobacterium: (i) co-cultivation of Agrobacterium with cultured isolated protoplasts, (ii) transformation of cells or tissues with Agrobacterium, or (iii) transformation of seeds, apices or meristems with Agrobacterium. The first method requires an established culture system that allows culturing protoplasts and plant regeneration from cultured protoplasts. The second method requires that the plant cells or tissues can be transformed by Agrobacterium, and that the transformed cells or tissues can be induced to regenerate into whole plants, while the third method requires micropropagation.

Transgenic cells, e.g., callus cells, are broken into small clumps of typically 1-20 or more cells, and suspended in a suitable cell culture medium. The cells are preferably cultured under conditions that favor plant cell growth, until the cells reach a desired cell density, then under conditions that favor expression of the target gene under the control of the appropriate promoter.

A typical transformation protocol for rice generally follows the methods detailed generally in Sivamani et al., 1996; Zhang et al., 1996; and Li, et al., 1993. Briefly, seeds are sterilized by standard methods, and callus induction from the seeds is carried out on MB media with 2,4D. During a first incubation period, callus tissue forms around the embryo of the seed. By the end of the incubation period, (e.g., 14 days at 28° C.) the calli are about 0.25 to 0.5 cm in diameter. Callus mass is then detached from the seed, and placed on fresh NB media, and incubated again for about 14 days at 28° C. After the second incubation period, satellite calli develop around the original “mother” callus mass. These satellite calli are slightly smaller, more compact and defined than the original tissue, and are transferred to fresh media.

Calli to be bombarded are selected from 14 day old subcultures. The size, shape, color and density are all important in selecting calli for transformation. The calli typically should be between about 0.8 and 1.1 mm in diameter and appear as spherical masses with a rough exterior.

After transformation preferably by particle bombardment, the cells are typically grown under conditions that favor selection of transformants in the presence of the substrate for the selectable marker, e.g., hygromycin, the bialophos resistance gene or phosphinotricin. Alternatively, plasmid pEmuGN (Last, 1991), which directs high level expression of β-glucoronidase in monocots, can be used as an internal control to monitor the efficiency of gene transfer; substrate 5-bromo-4-chloro-3-indolyl is then used for histochemical staining of transformed tissue.

Preferably, the transformed cells are cultured under multiple rounds of selection to produce a uniform, stably transformed cell line.

Reproducible transformation frequencies are typically obtained by growing the plants over a period of 60-80 days at temperature cycles of 18° C. for 16 h and 12° C. for 8 h using high light intensities above about 120 μE/m2.

A preferred transformation protocol for monocots is as follows. Immature embryos of about 1.5-2.5 mm in length are isolated from plants and grown under controlled conditions (day 0). The embryos are then bombarded with the target chimeric gene containing a selectable marker, e.g., a herbicide resistance gene (day 1) and placed on selection media (day 2). During week 2, a first subcultivation is carried out and most immature embryos have exhibited growth. A second subcultivation is carried out at week 4, followed by a third subcultivation during week 6. During week 6, first callus is placed on shoot inducing medium. At about week 8, a fourth subcultivation is carried out and the first clones are placed on regeneration media. At week 10, a fifth subcultivation is carried out, and clones are placed on shoot inducing medium. By about week 12, a sixth subcultivation is conducted on shoot inducing and regeneration media; typically, the first green plantlets appear during this stage. At about week 14, a seventh subcultivation is carried out; this is followed during week 16 by an eighth subcultivation. During this period the first clones are then transferred to soil and allowed to grow for up until about month 7. Nucleic acids are then isolated from plant tissue and analyzed by PCR for the presence of the chimeric gene and the metabolic products impacted by altered expression of that the target gene. By about month 8, the T0 plants begin setting grains, and during month 9, the immature grains are harvested and the T1 embryos germin.ated to establish the next generation. The T1 plants are grown to maturity, and the success rate of the transformation determin.ed. Typical rates of transformation are about 1% transformants per isolated immature zygotic embryo.

Plant regeneration from cultured protoplasts or callus tissue is carried by standard methods, e.g., as described above and in Evans et al., HANDBOOK OF PLANT CELL CULTURES, Vol. 1: (MacMillan Publishing Co. New York, 1983); and Vasil I. R. (Ed.), CELL CULTURE AND SOMATIC CELL GENETICS OF PLANTS, Acad. Press, Orlando, Vol. I, 1984, and Vol. III, 1986.

In a preferred method for producing plants with elevated levels of G-lignans in seeds, monocot plant cells are transformed and used to regenerate plants, seeds from the plants are harvested, germin.ated, and the seeds derived from such transformed plants are processed as appropriate for use as food additives.

Following introduction of the chimeric gene and growth of transformants, transformation of plant tissue can be confirmed by a variety of methods. Exemplary methods for confirming transformation and for analyzing proteins, enzymes and metabolites in seeds derived from transgenic monocot plants, are described below.

VI. EVALUATION OF TRANSGENIC PLANTS

A. PCR ANALYSIS

DNA may be extracted from various plant tissues, particularly seed tissues or whole seeds and analyzed for the presence of a target gene by use of appropriate primers. (See, e.g., Jensen, et al., 1996; FIG. 15).

B. RT PCR ANALYSIS

RNA is extracted from various plant tissues, particularly seed tissues and from whole seeds, reverse transcribed and is analyzed for the presence of the mRNA corresponding to the target gene. mRNA from the same tissues is also analyzed for sequences encoding downstream proteins or enzymes integral to the phenylpropanoid pathway leading to G-lignans for which altered expression is expected based on modified expression of the target gene, by use of appropriate primers (Jensen et al., 1996). In addition, if expression of dirigent protein is up-regulated, analyses may include e.g., an evaluation of the expression of secoisolariciresinol dehydrogenase (SD) in the same tissues.

C. SOUTHERN ANALYSIS

Transformation of each plant can be confirmed using Southern blot analysis of genomic DNA. Typically, total DNA is isolated from each transformant (e.g., Schwarz-Sommer, et al., 1984). The DNA is then digested with restriction enzyme, fractionated in 1% agarose gels and transferred to nylon filters (e.g., HYBOND-N, Amersham) according to standard techniques. The blot may then be probed, e.g., with ³²P-labeled target cDNA as described.

D. NORTHERN ANALYSIS

RNA is isolated from specific seed tissues (e.g., seed testa layer, pericarp, aleurone and endosperm) or at specific developmental stages, separated, e.g., in a 1.2% agarose gel containing 2.2M formaldehyde, and blotted to a nylon filter, e.g., Hybond-N, according to the supplier's protocol. Strand specific RNA probes are synthesized by phage T7 and T3 RNA polymerases from the Ant18 cDNA clone and hybridized to the RNA on the filter. This allows an estimation of the amount of endogenous sense and transgenic antisense mRNA for the target enzyme or protein.

E. HIGH PERFORMANCE LIQUID CHROMATOGRAPHY (HPLC)

Extracts from seeds of transformed and native plants may be analyzed by high performance liquid chromatography (HPLC), in order to detect the presence and/or levels of particular intermediates in the phenylpropanoid pathway, including, but not limited to L-phenylalanine, cinnamate, para-coumarate, caffeate, ferulate, sinapate, para-coumaryl CoA, caffeoyl CoA, feruoyl CoA, 5-hydroxy feruoyl CoA, sinapoyl CoA, para-coumarylaldehyde, coniferylaldehyde, 5-hydroxy coniferylaldehyde, sinapylaldehyde, para-coumaryl alcohol, coniferyl alcohol, and sinapyl alcohol.

F. ENZYME ASSAYS

Enzyme extracts of seeds from monocot plants can also be assayed for activity of the various enzymes integral to the phenylpropanoid pathway. For example, phenylalanine amin.o lyase (PAL), cinnamate-4 hydroxylase (C4H), 4-coumaric acid coenzyme A ligase (4Cl), caffeate acid O-methyl transferase (C-OMT), ferulate-5-hydroxylase (F5H), chalcone synthase (CHS), coumaroyl-coenzyme A 3-hydroxylase (CCoA-3H), caffeoyl-CoA 3-O-methyltransferase (CCoA-OMT), cinnamoyl-coenzyme A reductase (CCR), pinoresinol/lariciresinol reductase (PR), secosisolariciresinol dehydrogenase (SD) and secosisolariciresinol diglucosyl transferase (SDT) may be analyzed.

G. LIGNAN ANALYSIS

In one exemplary approach, G-lignans are analyzed by carrying out the steps of (1) separating the appropriate plant tissues into components for comparative analysis, (2) releasing the lignans from the plant material by the action of hydrolytic enzymes and acid, (3) extraction of lignans with diethyl ether, (4) purification on DEAE cellulose and QAE Sephadex columns, silylation and (4) quantitation by gas chromatography/mass spectroscopy using isotope dilution and selected ion monitoring (SIM; Nilsson et al., 1997; Mazur et al.,1996).

An analysis of rye following roller-milling into 6 flour fractions, a short and a bran, indicated the highest concentrations of the G-lignans, secoisolariciresinol and matairesinol, in the short and the bran, indicating that the majority of both lignans are present in the outer layers of the rye kernal, however lower levels of secoisolariciresinol also appeared to be present in the starchy endosperm (Nilsson et al., 1997).

In practicing the present invention, extracts taken from seeds of transformed and native plants may be analyzed by gas chromatography, together with mass spectroscopy using isotope dilution and selected ion monitoring as further described below, in order to detect the presence and/or levels of particular lignans such as matairesinol or secoisolariciresinol diglucoside. Detection of, or an increase in, the level of matairesinol or secoisolariciresinol diglucoside in transformed plants relative to native (non-transformed) plants is an indication that introduction and stable expression of the chimeric gene construct has resulted in modifying the level of G-lignans in the transformed plants.

H. ELEVATED LIGNAN CONTENT IN SEEDS AND SEED PRODUCTS

Secoisolariciresinol and matairesinol mainly occur in the form of glycosides in plants. After hydrolysis of the glycosides, the concentrations of secoisolariciresinol and lariciresinol have been evaluated in various soybean products, grains and other foods. (Adlercreutz et al., 1997).

The highest levels of secoisolariciresinol reported to date have been those detected in flaxseed and pumpkin seed (approximately 370- 545 mg/100 g and 21 mg/100 g, respectively). See, e.g., Thompson, 1998; Adlercreutz et al., 1997. In addition, moderate levels of secoisolariciresinol have been detected in numerous grains examples of which include; whole grain wheat, wheat bran, oat meal, oat bran, whole grain barley, barley bran, whole grain rye, rye bran, whole grain triticale, and triticale meal (approximately 33, 110, 13, 24, 58, 63, 47, 132, 39, 21 μg/ml, respectively; Adlercreutz et al., 1997). Further, secoisolariciresinol has also been found to be present in a diverse list of food products including, but not limited to, soybean flour, sunflower seeds, poppy seeds, caraway seeds, mung beans, mung bean sprouts, urid dahl beans, carrots, broccoli, garlic, cranberries, peanuts, Earl Grey Black tea, Japanese green tea, etc. (approximately 130, 610, 14, 221, 172, 468, 240, 192, 414, 379, 1510, 333, 1590, and 2460 μg/ml, respectively; Adlercreutz et al., 1997).

The corresponding level of matairesinol in flaxseed has been determin.ed to be (1090-1300 μg/ml; Adlercreutz et al., 1997). Levels of matairesinol in various grains exemplified by whole grain wheat, oat bran, whole grain rye, rye bran, whole grain triticale, and triticale meal were determin.ed to be approximately 3, 155, 65, 167, 9, and 11 μg/ml, respectively (Adlercreutz et al., 1997). The amount of matairesinol in poppy seeds, caraway seeds, mung bean sprouts, urid dahl beans, carrots, broccoli, garlic, Earl Grey Black tea, Japanese green tea, has been determin.ed to be approximately 12, 6, 1, 79, 3, 23, 4, 197 and 186 μg/ml, respectively (Adlercreutz et al., 1997).

In contrast to secoisolariciresinol, matairesinol has not been detected in pumpkin seeds, wheat bran, whole grain barley, barley bran, soybean flour, sunflower seeds, or cranberries, and has been found in only trace amounts in oatmeal, mung beans, and peanuts. (Adlercreutz et al., 1997). No correlation between the concentrations of secoisolariciresinol and matairesinol was found in whole meal rye flour (Nilsson et al., 1996).

The methods and compositions of the present invention reflect an alteration in G-lignan concentration in the seeds of monocot plants, particularly the G-lignans secoisolariciresinol diglucoside and matairesinol. The introduction of the chimeric gene constructs of the present invention and the corresponding expression of proteins integral to the phenylpropanoid pathway in monocot seeds, yield a change in the concentration of G-lignans over the concentration of G-lignans found in the seeds of monocot plants that have not been transformed with such chimeric gene constructs (i.e. plants in their native state).

Ordinarily, a “change in lignan concentration or lignan level” will correspond to at least about a 2× increase in the G-lignan concentration or level detectable in the seeds of transformed plants relative to the seeds of plants that have not been transformed with the chimeric gene constructs of the present invention. Such increases in G-lignan concentration are preferably at least about 5×, and more preferably at least about 10× or more relative to the G-lignan concentration present in the seeds of transformed plants relative to the seeds of plants that have not been transformed with the chimeric gene constructs of the present.

It will be understood that the invention provides plants seeds having elevated levels of G-lignans and that such seeds may be used in their native state, and may serve as the basis for seed extracts and food additives. Such seed extracts and food additives may be prepared using methods generally employed by those of skill in the art.

VII. UTILITY/BIOLOGICAL EFFECTS OF LIGNANS

Dietary consumption of processed transgenic seeds containing increased levels of G-lignans can result in increased levels of mammalian lignans (enterodiol and enterolactone) which are formed by bacterial metabolism of plant lignans contained in the diet. (Adlercreutz et al., 1997; Lampe et al., 1999.)

Plant lignans and have been suggested as capable of impacting a wide variety of biological processes (Adlercreutz et al., 1997). For example, many plant lignans have been shown to have anti-viral, anti-bacterial and fungistatic activitie; a lignan with close structural similarity to matairesinol has been found to have immunomodulatory activity; lignans have been shown to effect intracellular and steroid metabolic enzymes; protein synthesis; numerous anti-cancer effects, inhibition of chemotaxis, inhibition of multiple steps of the cell cycle of vascular smooth muscle cells and inhibition of endothelial cell proliferation. (See, e.g., Pool-Zobel B L et al., 2000; Li, et al., 1999; Adlercreutz, 1997; Ayres et al., 1990).

Epidemiological studies suggest that a lignan-rich diet such as that of American macrobiotics and vegetarians may be correlated with reduced incidence of breast cancer and protective effects during the promotional phase of prostate cancer.

The mammalian lignans along with the isoflavenoids are classified as phyto-oestrogens (Adlercreutz, 1997). The phyto-oestrogens are the hormone-like metabolites produced by metabolic processing of lignans and isoflavenoids, which have the ability to bind with low affinity to oestrogen receptors as well as having weak oestrogen activity (Schutt, 1972; Setchell, 1988). Enterolactone, the most abundant mammalian lignan competes with the natural substrate for placental aromatase. Placental aromatase converts its natural substrate androstenedione, into oestrone which has been implicated in various types of cancers.

The lignans and isoflavenoids appear to stimulate sex hormone-binding globulin (SHBG) synthesis in the liver and may in this way influence the biological effects of sex hormones. Phyto-oestrogens have been implicated in inhibition of numerous biosynthetic and metabolic enzymes, and such activities are a likely source of their disease-preventive effects. (See, e.g., Adlercreutz et al., 1997.)

From the foregoing, it will be appreciated that the invention provides methods for increasing the concentrations of G-lignans in the seeds of monocots. The invention further provides plants and seed compositions produced using such methods. The following examples are intended to illustrate, but in no way limit the invention.

MATERIALS AND METHODS

A. TRANSFORMATION AND ISOLATION OF POSITIVE COLONIES

40 μl of competent cells (DH10B) are placed on ice and 1-2 μl of ligated plasmid DNA added followed by gentle mixing and allowing the mixture to stand on ice for 2 min.. The cells are then electroporated and 1 ml of SOC medium immediately added, followed by incubation at 37° C., 225 rpm for 45 min.. 25 μl, 50 μl and 100 μl of culture cells are spread on LB medium plates containing 0.1 mg/ml Ampicillin and incubated at 37° C., 225 rpm, overnight.

B. SMALL-SCALE CULTURES (MIN.IPREPS)

Small-scale cultures are prepared by picking several single-colonies and inoculating them into 3 ml of LB medium containing 0.1 mg/ml ampicillin and incubating at 37° C., 225 rpm, overnight. 500 μl of culture cells are stored in glycerol for large-scale culture and 60 μl of plasmids are isolated following conventional methods, then digested with restriction enzymes to confirm the presence of the lignan gene.

C. LARGE SCALE CULTURES (MAXIPREPS)

Large scale cultures of expression vectors (volume of 500 ml) were prepared for rice transformation starting with 300 μl of min.iprep-culture cells containing the correct plasmid. For future use, 700 μl of grown cells were added to 300 μl of 50% glycerol and stored in −70° C.

D. LIGNAN ANALYSIS

D1. CHEMICALS AND REAGENTS.

Methanol and n-hexane (HPLC grade, glacial acetic acid, NaOH, Et2 O, ascorbic acid, hydrochloric acid were obtained from Aldrich. Tri-Sil reagent and Silyl-8 were from Pierce Chemical Co. Helix pomatia juice was purchased from Biosepra, Inc.

D2. CHROMATOGRAPHIC MATERIAL.

DEAE- and QAE-Sephadex A-25 are washed with 20%, 50% and absolute ethanol and stored at 6° C. The hydroxyl form of DEAE-Sephadex A-25 is obtained by washing the gel successively with 0.1M NaOH in 70% CH₃OH, 70% CH₃OH and then CH₃OH. The DEAE-Sephadex A-25 is used immediately after conversion. The acetate form of QAE-Sephadex A-25 is obtained by washing the gel successively with 0.1M NaOH in 70% CH₃OH, 70% CH₃OH, 0.5M Acetic Acid in 70% CH₃OH, 70% CH₃OH and then CH₃OH. The acetate form of QAE-Sephadex A is stored at 6° C.

D3. STANDARDS

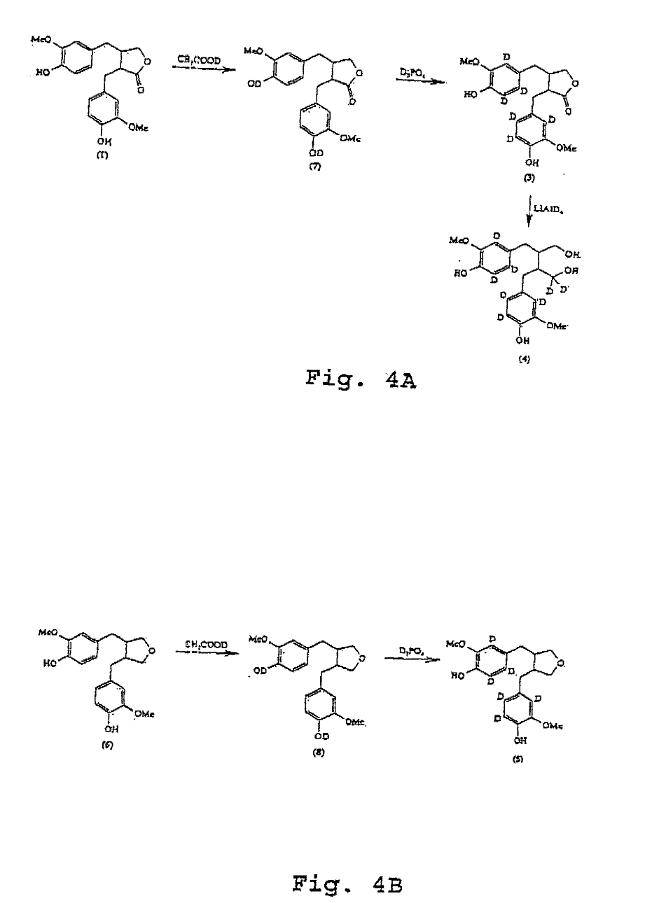

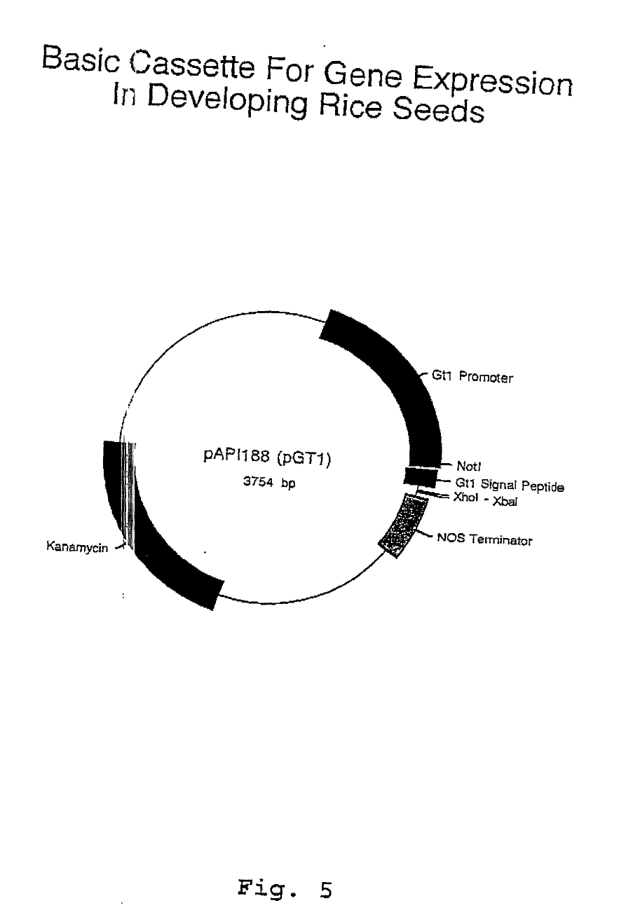

Natural abundance matairesinol (1), secoisolariciresinol (2), and anhydrosecoisolariciresinol (6) were previously synthesized according to known methods. Deuterated d6-matairesinol (3), d8- secoisolariciresinol (4) and d6-anhydrosecoisolariciresinol (5) were synthesized as described below. (See FIGS. 4A-4B)

D4. SYNTHESIS OF D6-MATAIRESINOL (3)

Matairesinol (1) (0.356 g, 0.99 mmol) was dissolved in deutero-labeled acetic acid. After several hours at room temperature, the solution was evaporated in vacuo to small volume to give (FIG. 4A). Deutero-labeled acetic acid was made from freshly distilled acetic anhydride (32.5 g, 0.32 mol) to which heavy water (8.8 g, 0.44 mol) was slowly added with stirring under argon for 4 h. Predeuterated matairesinol was added under argon to labeled phosphoric acid, which was prepared by adding D₂O (6 g) to P₂O₅ (6 g). The reaction mixture was stirred at 80° C. for 3 days. Water (10 ml) was next added to the cooled reaction mixture, which was then extracted with ether (15 ml), followed by washing the ether layer with NaHCO₃ (15 ml×2) and water (15 ml×2). The organic phase was dried with MgSO4 and evaporated to dryness. The above deuteration and work-up were repeated once again. The crude product was recrystallized from n-hexane-EtOAc to give (3; FIG. 4A).

Spectral data (3; FIG. 4A) includes the following: H-NMR_(CDCl3): 6.80 -6.40 (6H, m, Ar-H), 5.42 (2H, s, OH×2), 4.16 and 3.90 (1H,m, H-9, H-9′), 3.82 (3H, s, OCH3), 3.81 (3H, s, OCH3 ), 2.92 (2H, m, H7′), 2.55 (4H, m, H-7, H-8, H-8′); EI-MS: m/z 358 (M+), 136. (4) 1 H-NMR_(CDCl3): 5.50 (2H, s, OH×2), 4.17 and 3.90 (1H, m, H-9, H9′), 3.81 (3H, s, OCH3), 3.82 (3H, s, OCH3), 2.93 (2H, m, h-7′), 2.57 (4H, m, H-7, H-8, H-8′); EI-MS: m/z 364 (M+), 140.

D5. SYNTHESIS OF D8-SECOISOLARICIRESINOL (4)

d6-Matairesinol (3) (120 mg) was dissolved in tetrahydrofuran (THF, 9 ml) freshly distilled over LiAIH₄ under N₂. The resulting solution was added dropwise, at room temperature, over a period of 15 min. to a stirred suspension of LiAID₄ (106 mg) in dry THF (2.4 ml). Following stirring for an additional hour at the same temperature, the reaction mixture was cooled to 0° C. Next, EtOAc (3 ml) was added dropwise, and the whole mixture poured onto dry ice. Distilled water (5 ml) was added to the resulting suspension, with the organic solvent removed in vacuo. The sample was reconstituted in distilled water (10 ml) with the whole extracted EtOAc (20 ml×6). The combined EtOAc solubles were washed with a saturated NaCl solution, dried with anhydrous MgSO4 and evaporated in vacuo to yield crude d8-secoisolariciresinol, which was purified by preparative TLC (silica gel) to give d8-secoisolariciresinol (4; FIG. 4A).

Spectral data (4; FIG. 4A) includes the following: H-NMR_(CD3 OD): 6.71 -6.44 (6H, m, Ar-H), 4.05 (2H, dd, H-9, H-9′), 3.70 (3H×2, s, O-CH3), 3.58 (2H, dd, H-9, H-9′), 2.65 (2H, dd, H-7, H-7′), 1.86 (2H, m, H-8, H-8); EI-MS: m/z 362 (M+), 189, 137. (4). 1 H-NMR_(CD3 CN): 3.74 (6H, s, OCH3 ), 3.43-3.32 (2H, m, H-9, H-9′), 2.88 and 2.65 (2H, d, J=8Hz, H-7, H-H-7′), 1.87 (2H, m, H-8, H-8′); EI-MS: m/z 370 (M+), 140.7.

D6. SYNTHESIS OF D6-ANHYDROSECOISOLARICIRESINOL (5)

Anhydrosecoisolariciresinol (40 mg) (6) was dissolved in deutero-labeled acetic acid. After several hours at room temperature, the solution was evaporated in vacuo to small volume to give (8). Deutero-labeled acetic acid was made from freshly distilled acetic anhydride (3.65 g, 35 mmol) to which heavy water (0.98 g, 49 mmol) was slowly added with stirring under argon 8 h. Predeuterated anhydrosecoisolariciresinol (8) was added, under argon, to deutero-labeled phosphoric acid, which was prepared by adding D2 O (1 g) to P2O5 (1 g). The reaction mixture was stirred at 80° C. for 3 days. Water (5 ml) was added to the cooled reaction mixture, which was then extracted with ether (5 ml×6) and washed with NaHCO₃ solution (5 ml×2) and water (5 ml×2). The organic phase was dried with MgSO₄ and evaporated to dryness. The above deuteration and work-up was repeated once again to give a residue (35.2 mg), which was purified by preparative TLC (silica gel) to give (5, FIG. 4). The crude product was recrystallized from n-hexane-EtOAc.

Spectral data (6, FIG. 4) includes the following: 1H-NMR_(CDCl3): 6.82 (2H, d, J=8 Hz, H-5, H-5′), 6.60 and 6.57 (4H, m, H-6, H-6′), 6.5 (2H, br. s, H-2, H-2′), 5.51 (2H, br. s, OH×2), 3.83 (3H, s, OCH3), 3.82 (3H, s, OCH3), 3.92 and 3.54 (2H, dd, H-9, H-9′), 2.55 (4H, m, H-7, H-7′), 2.19 (2H, m, H-8, H-8′); EI-MS: m/z 344 (M+),137.

Spectral data (5; FIG. 4) includes the following: 1H-NMR (CDCl3); _{—}5.50 (2H, br. s, OH×2), 3.82 (3H, s, OCH3), 3.81 (3H, s, OCH3), 3.92 and 3.54 (2H, dd, H9, H-9′), 2.55 (4H, m, H-7, H-7′), 2.19 (2H, m, H-8, H-8′) and showed the absence of aromatic protons; EI-MS: m/z 350 (M+), 137.

### EXAMPLE 1

### An Efficient Rice Transformation System With a Selectable Marker Driven by a Non-Constitutive Promoter

A. CONSTRUCTION OF A SELECTABLE MARKER PLASMID, PAPI76.

A selectable marker plasmid pAPI76 was constructed in three steps. First, a DNA fragment was amplified from a rice alpha-amylase gene, RAmy1A, (Huang et al., 1990) and cloned into pBluescript KS+ at the SmaI/EcoRI restriction sites. The primers used to amplify the fragment were 1AR1 (SEQ ID NO: 1) and 1Asma (SEQ ID NO:2). The amplified fragment contained 297 bp of RAmy1A termin.ator. This resulting plasmid was called p1AT. Second, a BamHI DNA fragment from pGL2 (Shimamoto et al., 1989) was cloned into BamHI site of p1AT. The BamHI fragment contained most of the hygromycin phosphotransferase (hph) gene with a deletion of four amin.o acids at the C-termin.us. This plasmid was named pAPI74. Third, a SacI/XbaI fragment amplified from a glucanase gene, Gns9, was inserted into pAPI74 cut with SacI/XbaI to form pAPI76. The primer sequences used to amplify the Gns9 promoter were GnsF (SEQ ID NO: 3) and GnsR (SEQ ID NO: 4). PCR fragments were confirmed by DNA sequencing. To visualize the tissue specific expression of the Gns9 promoter, a Gns9 promoter—GUS construct may be prepared for rice transformation. The GUS gene and nos termin.ator was obtained from pBI221 (ClonTech, CA) by digestion with restriction enzyme EcoRI and BamHI. The fragment was then inserted into pAPI76, which was cut with the same enzymes. Thus, the GUS gene replaced the hph gene, resulting in a plasmid pAPI83.

B. RICE TRANSFORMATION

The procedure for microprojectile-mediated rice transformation (Sivamani et al., 1996; Chen et al., 1998) was followed with modifications. Approximately 200 TP309 seeds were dehusked, sterilized in 50% commercial bleach for 25 min. and washed with sterile water three times for 5 min. each. The sterilized seeds were placed plates containing N6 semisolid medium supplemented with 2,4-D 2 mg/L, sealed with parafilm and incubated in the dark at 28° C. for 14 days to induce calli.

Developing calli (less than 1 mm in diameter) were then transferred developing onto fresh N6 medium (35 calli per plate) for 30 days and serve as bombardment material. Calli were subcultured every 15 days.

Prior to bombardment, calli from 30-day old subcultures are selected with size, shape, color, and density very important factors for successful transformation. It is preferred that calli for transformation have the following characteristics: a size of 1.0-3 mm in diameter, a spherical mass with a rough exterior, a white and opaque color and a density which is compact and hard.

Prior to transformation, approximately 200 calli are placed together in the center of a plate (about 4 cm in diameter) on N6 semisolid medium and incubated at 28° C. in the dark for 24 hours. The efficiency of rice co-transformation over eight plasmids is presented in Table 1, with the procedure further detailed in Example 3.

TABLE 1
EFFICIENCY OF RICE CO-TRANSFORMATION
OVER EIGHT PLASMIDS

| | Selectable | Selectable Marker Gene | | Target Gene | |
|---|---|---|---|---|---|
| Target | marker | PCR | PCR | PCR | PCR |
| plasmid | plasmid | positive | negative | positive | negative |
| pAPI65 | pAPI76 | 33 | 0 | 33 | 0 |
| pAPI72 | pAPI76 | 27 | 0 | 27 | 0 |
| pAPI96 | pAPI76 | 30 | 0 | 30 | 0 |
| pAPI85 | pAPI76 | 26 | 0 | 22 | 4 |
| pAPI98 | pAPI76 | 13 | 0 | 13 | 0 |
| PAPI90 | pAPI76 | 13 | 0 | 13 | 0 |
| pAPI64 | pAPI76 | 13 | 0 | 13 | 0 |
| pAPI78 | pAPI76 | 28 | 0 | 26 | 2 |
| TOTALS | | 183 | 0 | 177 | 6 |

### EXAMPLE 2

### Promoter Cassette for Tissue-Specific Expression of Lignan Genes in Rice.

A. EXPRESSION PLASMIDS CONTAINING LIGNAN BIOSYNTHESIS GENES EXPRESSED UNDER THE CONTROL OF THE ENDOSPERM-SPECIFIC GT1 PROMOTER

Four plasmids were generated and used to express genes involved in lignan biosynthesis in developing rice seed The plasmids contain the coding sequence for dirigent protein; laccase; pinoresinol/lariciresinol reductase; and secosisolariciresinol dehydrogenase under the control of the rice endosperm-specific glutelin (Gt-1) promoter: pGt-1-DIRG, dirigent protein; pGt-1-LACC, laccase; pGt-1-REDS pinoresinol/lariciresinol reductase; and pGt-1-DEHY secosisolariciresinol dehydrogenase.

In order to enhance the level of the plant lignans secoisolariciresinol and matairesinol in rice endosperm, four key proteins in the G-lignan biosyntheitc pathway were placed under the control of the rice glutelin 1 promoter, (Gt1) in preparing vector constructs for subsequent transformation.

Promoter cassettes were developed for expressing lignan genes in rice endosperm tissues using the endosperm-specific glutelin (Gt-1) promoter.

The glutelin 1 gene promoter was cloned using two primers based on the glutelin 1 (Gt1) gene sequence (Okita et al., 1989). The forward primer with a HindIII site was designated MV-Gt1-F1 (SEQ ID NO: 5), and the reverse primer was designated Xba-gt1-R1 (SEQ ID NO: 6). Crude DNA was isolated from leaves of the rice variety M202 and the PCR product amplified from the crude DNA cloned into pCR2.1 (Invitrogen, CA) in both orientations. The resulting plasmid was named pCRGT1 or pAPI134.

To generate the Gt1 expression cassette plasmid, pAPI134 was digested with Hind III and Xba I and the fragment containing Gt1 promoter was cloned into pAPI135, which contains the nos termin.ator. The resulting plasmid, pAPI141, contained the Gt1 promoter, the Gt1 signal sequence, a multiple cloning site and a nos termin.ator. In order to make a NotI site at the 5′ UTR of Gt1, two primers were generated. The first primer is Gt1SDMF (SEQ ID NO: 34), and the second primer is Gt1SDMR (SEQ ID NO: 35). The protocol from Stratagene's Quickchange kit, using pAPI141, Gt1SDMF and Gt1SDMR, was followed to create the NotI site. The resulting plasmid was named pAPI188(Kan). The mutation was confirmed by sequencing.

The coding sequences for selected genes which encode proteins integral to the lignan biosynthetic pathway are presented in FIGS. 7A-C, 8A-C, 9A-C, and 10A-C. The coding sequences were full length for dirigent protein; laccase; pinoresinol/lariciresinol reductase (reductase); while the coding sequence for secosisolariciresinol dehydrogenase (dehydrogenase), lacks at least the Met codon.

1. CLONING THE LACCASE GENE (LACC) INTO AN EXPRESSION CASSETTE

Two oligonucleotide primers, LACCF (SEQ ID NO: 9) and LACCR (SEQ ID NO: 10) were synthesized based on the laccase gene sequence. LACCF contains a NotI site while LACCR contains an XbaI site. The 1745 bp PCR product generated from the two primers was digested with NotI and XbaI. The processed PCR products were then ligated to the pAPI188 vector, prepared as described above. After ligation, transformation and min.i-screening of colonies, a positive clone was identified and the junction sequence between the vector and the gene were confirmed as correct. A restriction map of the plasmid (FIG. 6B), its DNA sequence and the coding sequence and corresponding amin.o acid sequence for laccase are presented in FIGS. 7A-C (SEQ ID NO: 11 and SEQ ID NO: 12, respectively).

2. CLONING THE DIRIGENT PROTEIN GENE (DIRG) INTO AN EXPRESSION CASSETTE

Two oligonucleotide primers, DIRGF (SEQ ID NO: 13) and DIRGR (SEQ ID NO: 14) were synthesized based on the dirigent protein gene sequence. DIRGF contains NotI site while DIRGR contains XhoI site. The 612 bp PCR product generated from these two primers was digested with NotI and XhoI. The processed PCR products were then ligated to the pAPI188 vector, prepared as described above. After ligation, transformation and min.i-screening of colonies, a positive clone was identified and the junction sequence between the vector and gene were confirmed as correct. A restriction map of the plasmid (FIG. 6A), its DNA sequence and the coding sequence and corresponding amin.o acid sequence for dirigent protein are presented in FIG. 8A-C (SEQ ID NO: 15 and SEQ ID NO: 16, respectively).

3. CLONING THE PINORESINOL/LARICIRESINOL REDUCTASE GENE (REDS) INTO AN EXPRESSION CASSETTE

Two oligonucleotide primers, REDSF (SEQ ID NO: 17) and REDSR (SEQ ID NO: 18) were synthesized based on the reductase gene sequence. REDSF contains NotI site while REDSR contains a SalI site. The 1013 bp PCR product generated from these two primers was digested with NotI and SalI. The processed PCR products were then ligated to the pAPI188 vector digested with NotI and XhoI (The ends generated by SalI and XhoI are compatible). After ligation, transformation and min.i-screening of colonies, a positive clone was identified and the junction sequence between the vector and the gene were confirmed as correct. A restriction map of the plasmid (FIG. 6C), its DNA sequence and the coding sequence and corresponding amin.o acid sequence for pinoresinol/lariciresinol reductase are presented in FIGS. 9A-C (SEQ ID NO: 19 and SEQ ID NO: 20, respectively).

4. CLONING THE SECOISOLARICIRESINOL DEHYDROGENASE (DEHY) GENE INTO AN EXPRESSION CASSETTE

Two oligonucleotide primers, REHYF (SEQ ID NO: 21) and DEHYR (SEQ ID NO: 22) were synthesized based on the dehydrogenase gene sequence. DEHYF contains NotI site whileDEHYR contains XhoI site. The forward primer contains ATG start codon which is followed by the GCC ACT codons. According to Lewis Lab, the truncated dehydrogenase produced in E coli showed correct function. The 886 bp PCR product generated from these two primers was digested with NotI and XhoI. The processed PCR products were then ligated to the pPAI188 vector, prepared as described above. After ligation, transformation and min.i-screening of colonies, a positive clone was identified and the junction sequence between the vector and the gene were confirmed as correct. The restriction map of the plasmid (FIG. 6D), its DNA sequence and the coding sequence and corresponding amin.o acid sequence for secoisolariciresinol dehydrogenase are presented in FIG. 10A-C (SEQ ID NO: 23 and SEQ ID NO: 24, respectively).

Table 2 presents characteristics of the plasmids used to express lignan biosynthesis genes under the control of the endosperm-specific Gt-1 promoter.

TABLE 2
PLASMIDS FOR EXPRESSION OF GENES
INVOLVED IN LIGNAN BIOSYNTHESIS.

| | Pro- | | | Diagnostic | Diagnostic |
|---|---|---|---|---|---|
| Plasmid | moter | Gene | Termin.ator | Enzyme | Fragment (bp) |
| pAPI245 | Gtl | Laccase | Nos | EcoRI/ | 689/4666 |
| | | | | XhoI | |
| pPAI244 | Gtl | Dirigent | Nos | NotI/XbaI | 594/3633 |
| pPAI246 | Gtl | Reductase | Nos | NotI/XbaI | 414/558/3633 |
| pPAI249 | Gtl | Dehydro- | Nos | NotI/XhoI | 866/3639 |
| | | genase | | | |

5. EXPRESSION PLASMIDS CONTAINING LIGNAN BIOSYNTHESIS GENES UNDER THE CONTROL OF THE ALEURONE-SPECIFIC CHITINASE GENE PROMOTER (CH126)

Promoter cassettes were also developed for expressing lignan genes in rice aleurone tissues under the control of the barley chitinase gene promoter (Chi26). The Barley chitinase 26 promoter was PCR-amplified from the genomic DNA of barley (Hymalaya), using primers Chi26FW (SEQ ID NO: 36) and Chi26RV (SEQ ID NO: 37). The resulting PCR product was digested with PstI and XbaI, and then used to replace the CaMV 35S promoter of pBI221.

Four plasmids were generated and used to express genes involved in lignan biosynthesis in developing rice seed. The plasmids contained the coding sequence for dirigent protein; laccase; pinoresinol/lariciresinol reductase; and secosisolariciresinol dehydrogenase under the control of the rice alleurone-specific Chitinase 26 (Chi26) promoter: pChi26-DIRG, dirigent protein; pChi26-LACC, laccase; pChi26-REDS pinoresinol/lariciresinol reductase; and pChi26-DEHY secosisolariciresinol dehydrogenase.

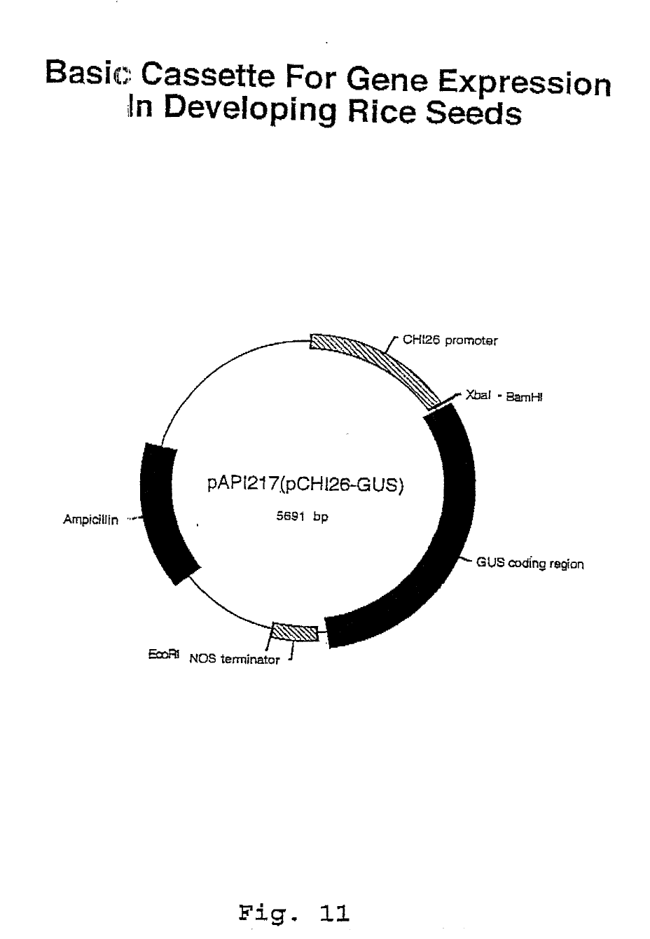

An expression cassette (pAPI217) was constructed containing the Chi26 promoter, the Nos termin.ator and multiple cloning sites between the two for easy cloning (FIG. 11). The coding sequences for the laccase, dirigent protein, reductase and dehydrogenase genes were removed from the Gt-1 plasmids API244 (pGt1-DIRG), pAPI245 (pGt1-LACC), pAPI246 (pGt1-REDS) and pAPI249 (pGt1-DEHY), and cloned into a Chi26 expression vector, pAPI217. Large scale preparations of each vector were made.

Once positive clones were identified and confirmed by restriction digestion and DNA sequencing, large scale preparation of plasmids was carried out using a Qiagen Plasmid Preparation Kit. The plasmids were confirmed to contain the correct fragments by restriction digestion as summarized below in Table 3.

TABLE 3
PLASMIDS FOR EXPRESSION OF
LIGNAN BIOSYNTHESIS GENES

| | Pro- | | Termi- | Diagnostic | Diagnostic |
|---|---|---|---|---|---|
| Plasmid | moter | Gene | nator | enzyme | fragments (bp) |
| pAPI260 | Chi26 | Laccase | Nos | EcoRI/ | 2011/3524 |
| | | | | Xbal | |
| pAPI261 | Chi26 | Dirigent | Nos | XbaI | 604/3803 |
| pAPI262 | Chi26 | Reductase | Nos | XbaI | 414/568/3803 |
| pAPI263 | Chi26 | Dehydro- | Nos | XbaI | 882/3803 |
| | | genase | | | |

Insert DNA was prepared by taking 5 μg of each DNA plasmid (pAPI244: pGt1-DIRIG, pAPI245: pGt1-LACC, pAPI246: pGT1-REDS and pAPI249: pGt1-DEHY) and digesting with 10 units of NotI at 37° C. for 1 h under standard reaction conditions.

Vector DNA was prepared by taking 5 μg of Chi26-Gus plasmid (pAPI217) and digesting with 10 units of BarnHI at 37° C. for 1 h under standard reaction conditions. Digested sites were filled in with T4 polymerase to generate a blunt end under standard reaction conditions.

The DNA was then extracted with chloroform by mixing and inverting several times, then centrifuging at 14,000 rpm for 5 min. (RT), followed by transferring the supernatant to a new tube, adding 50 μl of 7.5M NH4OAC, 300 μl of 100% chilled ethanol and inverting the tube several times, then immediately centrifuging at 14,000 rpm for 20 min. (4° C.). The supernatant was removed and 5001 μl of 70% cold ethanol added, followed by centrifuging at 14,000 rpm for 5 min.. The supernatant was removed and the DNA pellet air-dried. The pellet was dissolved in 35 μl of ddH2O and digested with EcoRI for 37° C. for 1 h. A 1% CTC agarose gel was run at 100V for 1 h using the undigested plasmid as a control. The DNA fragments were cut from the gels, put in a 1.5 ml centrifuge tube together with the same amount (v/w) of Buffer QG (QIA quick Gel Extraction Kit), then incubated at 50° C. for 10 min.. to melt the gel completely. The same volume of 100% cold isopropanol was ten added and the mixture spun down at 14,000 rpm for 1 min.., another 500 μl of QG buffer added, followed by pelleting at 14,000 rpm for 1 min.., addition of 750 μl of PE buffer and again pelleting at 14,000 rpm for 1 min.. 50 μl of EB buffer was then added to the center of filter tube, allowed to stand for 1 min., then pelleted at 14,000 rpm for 1 min.., followed by addition of 25 μl of 7.5M NH4OAC, 175 μl of 100% cold ethanol and immediately centrifuging at 15,000 rpm for 20 min.. (4° C,). The supernatant was removed and 500 μl of 70% cold ethanol added, followed by centrifuging at 14,000 rpm for 20 min.. The supernatant was removed and the DNA pellet air-dried, then dissolved in 10 μl of ddH2O.

50 ng of each insert DNA was ligated into 5 ng of vector DNA under standard reaction conditions overnight at room temperature. The following day, 5 μl of 7.5MNH4OAC and 35 μl of 100% cold ethanol was added, and the mixture was and immediately centrifuged at 15,000 rpm for 20 min. (4° C.). The supernatant was removed and 500 μl of 70% cold ethanol added, followed by centrifuging at 14,000 rpm for 5 min.. The supernatant was removed and the DNA pellet air-dried, then dissolved in 3 μl of ddH₂O.

### EXAMPLE 4

### Rice Transformation With Lignan Gene Expression Constructs Containing the GT1 Promoter or the CHI26 Promoter

To elevate the lignan concentration in transgenic rice seeds, eight high expression plasmids containing lignan biosynthesis genes were constructed including pAPI244 (pGt1-DIRG), pAPI245 (pGt1-LACC), pAPI246 (pGt1-REDS), pAPI249 (pGt1-DEHY), each with the lignan biosynthesis gene expressed under the control of the rice endosperm-specific glutelin (Gt-1) promoter which becomes active in endosperm during the rice seed maturation; and pAPI260 (pChi26-LACC), pAPI261 (pChi26-DIRG), pAPI262 (pChi26-REDS) and pAPI263 (pChi26-DEHY), each with the lignan biosynthesis gene expressed under the control of the Chi26 promoter or the barley Chitinase 26 (Chi26) gene promoter which becomes active in the alleurone layer during seed maturation.

A. RICE TRANSFORMATION AND PLANT REGENERATION

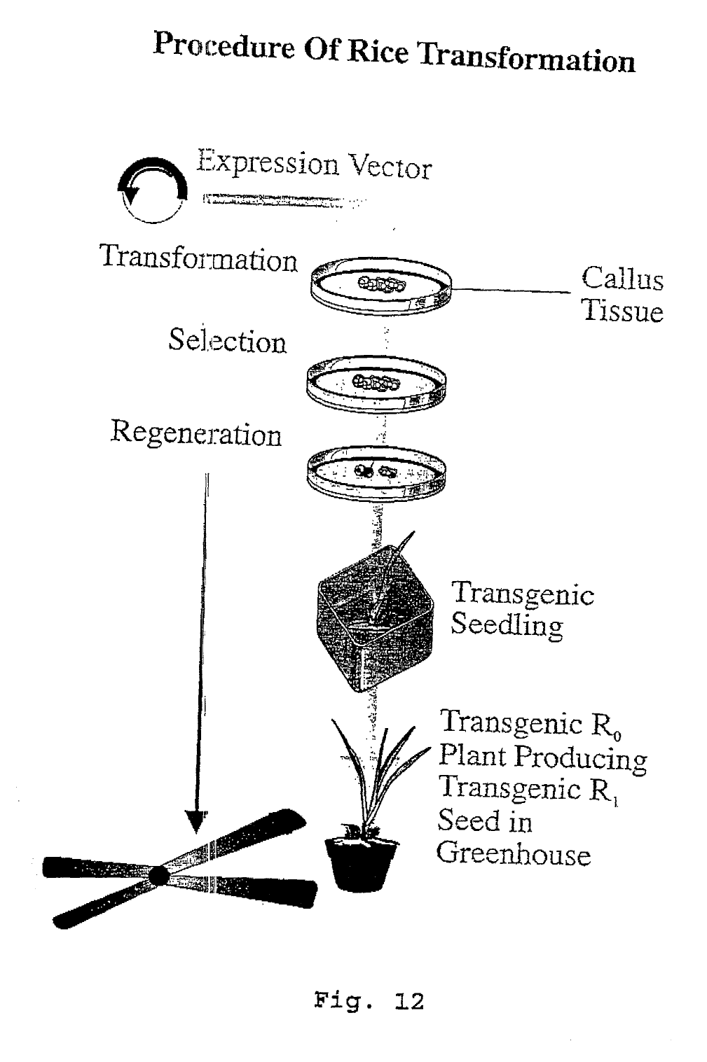

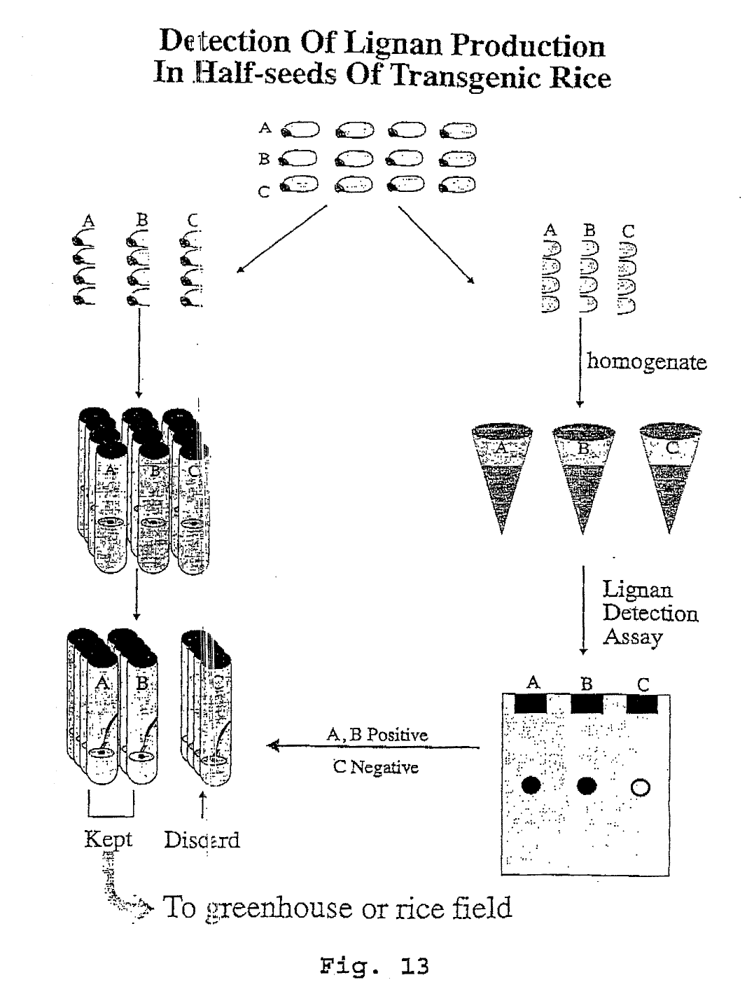

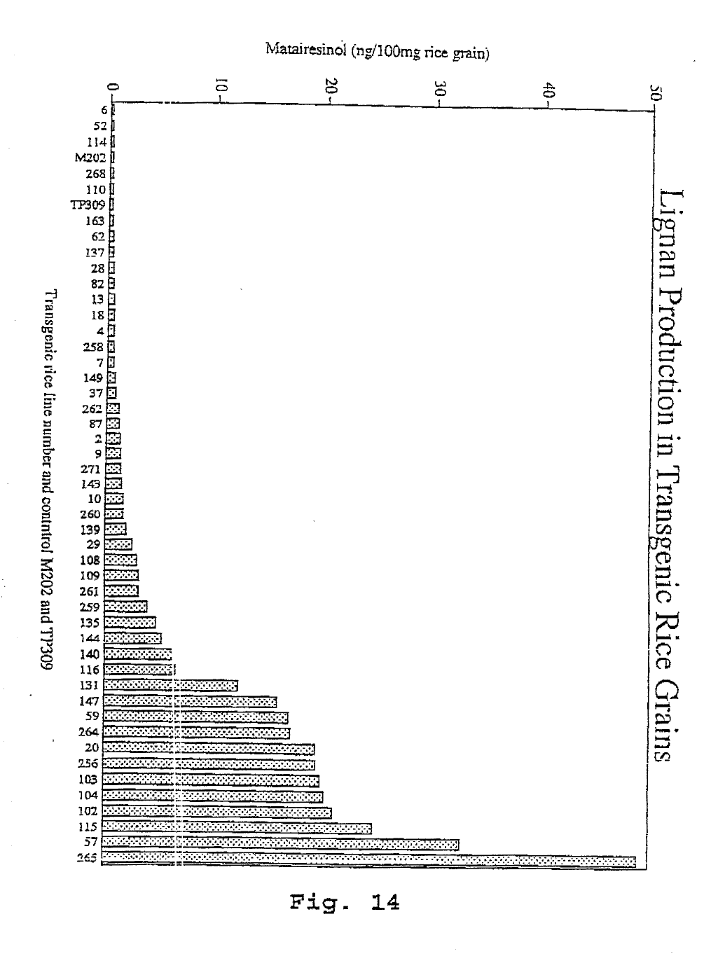

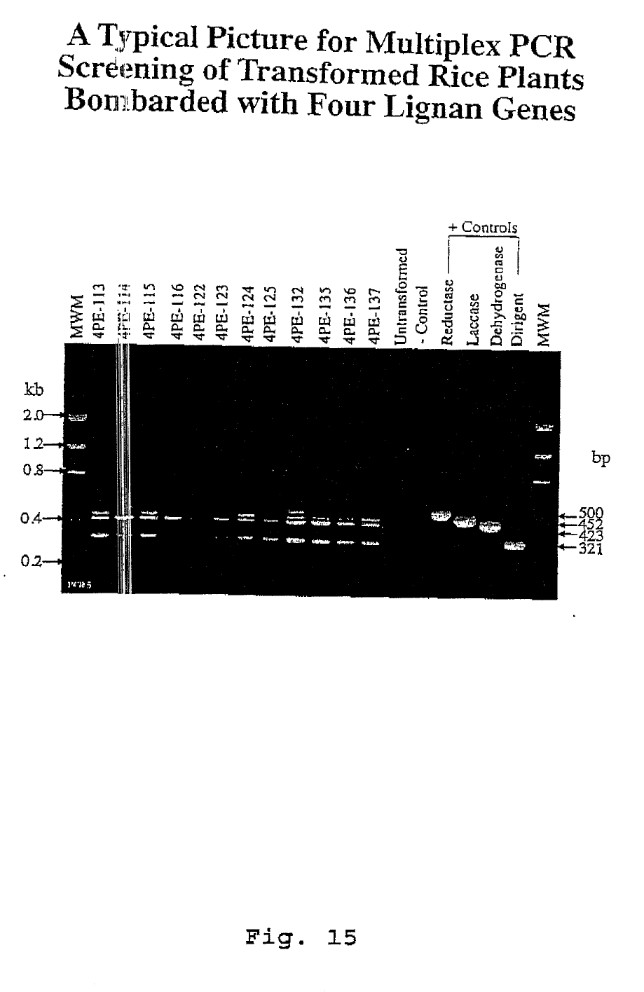

The procedure for microprojectile-mediated rice transformation (Sivamani et al., 1996; Chen et al., 1998) was followed with modifications. (See FIG. 12.) Approximately 200 TP309 seeds were dehusked, sterilized in 50% commercial bleach for 25 min. and washed with sterile water three times for 5 min. each. The sterilized seeds were placed plates containing N6 semisolid medium supplemented with 2,4-D 2 mg/L, sealed with parafilm and incubated in the dark at 28° C. for 14 days to induce calli.

Developing calli (less than 1 mm in diameter) were then transferred developing onto fresh N6 medium (35 calli per plate) for 30 days and serve as bombardment material. Calli were subcultured every 15 days.

Prior to bombardment, calli from 30-day old subcultures are selected with size, shape, color, and density very important factors for successful transformation. It is preferred that calli for transformation have the following characteristics: a size of 1.0-3 mm in diameter, a spherical mass with a rough exterior, a white and opaque color and a density which is compact and hard.

Prior to transformation, approximately 200 calli are placed together in the center of a plate (about 4 cm in diameter) on N6 semisolid medium and incubated at 28° C. in the dark for 24 hours.

60 mg 1.0 μM sized gold particles are weighed into a sterile 1.5 ml microcentrifuge tube, 1 ml of 100% EtOH added, followed by vortexing 2 min.utes at 1600 rpm. The particles are centrifuged for 10 seconds at full speed the supernatant discarded, then washed with 1 ml of sterile dd H2O 3 times, with vortexing for 2 min. at 1600 rpm each time, followed by centrifuging for 10 seconds and discarding the supernatant. The gold particles are resuspended in 1 ml sterile dd H₂O, and stored at −20° C. until used.

DNA from plasmids containing the Gtl promoter driven lignan genes: Reductase (PAPI 246), Laccase (pAPI 245), Dehydrogenase (pAPI 249), and Dirigent (pAPI 244); and the Chi26 promoter driven lignan genes: Reductase (pAPI 262), Laccase (pAPI 260), Dehydrogenase (pAPI 263) and Dirigent (pAPI 261) was prepared as described above and the concentration determin.ed for transformation of calli.

50 μl of washed gold particles were pipeted into a sterile 1.5 ml centrifuge tube, 5-20 μg of DNA added, the mixture is vortexed 3 seconds at 1300 rpm with 20 μl of 0.1M spermidine added while vortexing (1200 rpm) and 50 μl of 2.5M CaCl2 added in a dropwise fashion while vortexing, followed by vortexing an additional 10 min.utes, incubating at room temperature for 10 min.utes, centrifuging for 10 seconds, and discarding the supernatant. The DNA-gold particles were then resuspended in 20 μl of cold 100% ethanol and incubated on ice before loading onto microcarriers.

DNA-gold particles were loaded onto a microcarrier by pipeting 10 μl of DNA-gold particles onto the center (about 1 cm) of microcarriers, spreading as evenly as possible, then allowing them to air dry.

Biolistic bombardment was carried out with the Biolistic PDC-1000/He system (BIO-RAD). The procedure required 1.5 mg of gold particles (60 μg/μl) coated with 2.5 μg pAPI76 DNA and cotransfer plasmid DNA at a ratio of 1 to 6. DNA-coated gold particles were bombarded into the rice callus with a helium pressure of 1100 psi.

Exemplary bombardment conditions include: vacuum—27 inches Hg; rupture disk—1100 psi; Cell (target) position—8 cm from stopping screen; and two bombardments per plate.

After bombardment, the calli were allowed to recover on the same plate for 48 hrs and then transferred to N6 media containing 20 mg/l Hygromycin B. The bombarded calli were incubated on the selection media in the dark at 26° C. for 45 days. At this time, transformants were selected based on their white, opaque, compact appearance and transferred to pre-regeneration media consisting of N6 with 5 mg/l ABA, 2 mg/l BAP, 1 mg/l NAA and 20 mg/l Hygromycin B for 9 to 12 days. Transformants are easily distinguished from non-transformants which appear yellowish or brown, soft, and watery. The transformants were then transferred to regeneration media (RN) consisting of N6 (without 2,4-D) 3 mg/l BAP, and 0.5 mg/l NAA without Hygromycin B and cultured under continued lighting conditions for about two weeks.

When the regenerated plants were 1 to 3 cm high, plantlets were transferred to rooting media containing 0.05 mg/l NAA which was half the strength of MS media. After approximately two weeks in rooting media, plantlets developed roots shoots of 10 cm or more. The plants are then transferred to a 2.5 inch pot containing 50% commercial soil, Sunshine #1 (Sun Gro Horticulture Inc, WA) and 50% natural soil from rice fields. The pots are placed within a plastic container which is covered by another transparent plastic container to maintain 100% humidity and cultured under lighting conditions for 1 week, after which the transparent plastic cover was opened little by little during one day to gradually reduce the humidity. Afterwards, the plastic cover was removed completely, and water and fertilizers added as necessary. When the plants grew to approximately 12 cm tall, they were transferred to a greenhouse and allowed to grow to maturity. During potting, typically 2-3 cm of leaf tissue is taken for PCR analysis to determin.e whether the target genes were present in the plantlets.

In order to obtain large number of transformants, 173 plates of explants were bombarded with Gt1 constructs. After selection more than 3500 transgenic calli were selected and placed on regeneration medium, resulting in the generation of over 700 transgenic plants, and over 140 plant seed sets.

### EXAMPLE 5

### Analysis of Transgenic Rice Genomic DNA by PCR and Southern Blot

Hundreds of transgenic plants were produced by co-transformation of rice with the four lignan gene expression constructs. PCR analysis of seedling tissues from these plants indicated that many of the plants carried the lignan biosynthesis genes. The transgenic seedlings were transplanted into larger pots and moved to the greenhouse where they developed into healthy mature plants. An analysis of transgenic rice genomic DNA indicated that the majority of the plants carried multiple lignan biosynthesis genes.

A. PCR ANALYSIS OF TRANSGENIC PLANTS

Crude DNA suitable for PCR analysis was obtained using a simplified procedure was. In carrying out the method, about 2-3 cm of young leaf tissue was collected in a 1.5 ml eppendorf tube and the DNA extracted in the same tube and used as template DNA. The PCR reaction mixture contained about 50 ng of template DNA, 50 ng of each of primers, 0.05 mM dNTPs, 1× PCR buffer (10 mM Tris pH 8.4, 50 mM KC1, 2.0 mM MgCl2 , and 0.01 mg/ml gelatin) and 1 unit of Taq DNA polymerase in a volume of 50 μl. The template DNA was initially denatured at 94° C. for 2 min.utes followed by 28 cycles of PCR amplification with the following parameters: 30 seconds denaturation at 94° C., 90 seconds primer annealing at 58° C., and 90 seconds primer extension at 70° C. A final 5 min.utes incubation at 72° C. allowed for completion of primer extension. The amplified products were electrophoretically resolved on a 1.5% agarose gel in 1×TAE buffer.

Four pairs of PCR primers were designed to detect the presence of all four lignan genes in a single plant (Table 4). The resulting PCR reactions indicated that all four lignan biosynthesis genes could be simultaneously detected in a single PCR reaction (FIG. 15).

TABLE 4
PCR PRIMERS

| | | | |
|---|---|---|---|
| | | | Product |
| | | | Size |
| Promoter | Plasmid | Primers (5′ to 3′) | (bp) |

| | | | | |
|---|---|---|---|---|
| Gtl | Reductase | TCATTGGGGGTACAGGGTACTTAG | (SEQ ID NO:38) | 500 |
| | (pAPI 246) | TGCCAAATTGACAGAGACCTCC | (SEQ ID NO:39 | |
| Gtl | Laccase | TGCTAGTGCTTCCTCTTCATGCTGC | (SEQ ID NO:40) | 452 |
| | (pAPI 245) | CCCCTAATATGATTGTCGCTTCCGC | (SEQ ID NO:41) | |
| GtI | Dehydrogenase | GAGCCAGTGGAGTTGGAGAAGTC | (SEQ ID NO:42) | 423 |
| | (pAPI 249) | GCATGTGAAGAACCACCACCC | (SEQ ID NO:43) | |
| GtI | Dirigent | CAATGCCACTTCCGCCATAG | (SEQ ID NO:44) | 321 |
| | (pAPI 244) | CGCCATGAAAAAGTCACCAGTTCC | (SEQ ID NO:45) | |

The transformation for eight different lignan gene-containing plasmids was determin.ed and the results indicated that almost all the plants expressed the target gene and the selectable marker gene.

After hygromycin-based selection, 100 randomly chosen plantlets were analyzed by PCR to confirm the presence of those genes in rice cells. A comparison of positive (+control) and negative controls (untransformed/-control) revealed that out of 100 plants derived from transgenic calli 59 plants (59%) were positive for all four transgenes, 22 plants carried three genes, 7 plants were positive for two transgenes, 7 plants were positive for one transgene and 5 plants were negative for all four transgenes. Given that the PCR primers were targeted to a portion of each of the four transgenes, Southern blot analysis was carried out on the PCR positive plants to confirm that full length transgenes were present in the genome of those plants.

TABLE 5
PCR RESULTS

| | Plants | Plants | Plants | Plants | Plants |
|---|---|---|---|---|---|
| Plant # | with | with | with | with | with |
| analyzed | four genes | three genes | two genes | one gene | no genes |
| 100 | 59 (59%) | 22 (22%) | 7 (7%) | 7 (7%) | 5 (5%) |

B. SOUTHERN BLOT ANALYSIS OF TRANSGENIC PLANTS

About 3 gm of young leaves were collected from R0 transgenic plants and ground with liquid nitrogen into very fine powder. The powder was then placed in a 50 ml tube and 25 ml of extraction buffer (100 mM Tris-HCL, pH 8.0, 50 mM EDTA, pH 8.0, 500 mM NaCl, 1.25% SDS, 0.38% sodium bisulfite) was added. The mixture was incubated 15 min.utes at 65° C. with repeated shaking. Ten ml of 5M potassium acetate was added and the samples were incubated on ice for 20 min.utes with shaking. The tubes were centrifuged at 3000 rpm for 20 min.utes and the supernatant was filtered into another 50 ml tube through a miracloth. Two volumes of isopropanol were added and the tubes were kept at room temperature for 30 min.utes, then centrifuged at 3000 rpm for 10 min.utes. The DNA pellets were washed in 70% alcohol and resuspended in 5 ml of TE buffer (with RNase) and incubated at 37° C. for 30 min. Then 0.5 ml of 3M sodium acetate and 2 volumes of pre-chilled absolute alcohol were added to the tubes and incubated at 4° C. for 30 min. The DNA was hooked out, washed with 70% ethanol in a 1.5 ml tube and further resuspended in TE buffer. About 5 μg of HindIII/EcoRI digested DNA samples from each transgenic line were used to make a blot for Southern analysis.

The ECL™ direct nucleic acid labeling and detection system (Amersham) was used. Briefly, it involves directly labeling DNA with horseradish peroxidase by completely denaturing the probe so that it is in a single-stranded form. Then, peroxidase conjugated with a positively charged polymer is added and forms a loose attachment to the nucleic acid by charge attraction. Addition of gluteraldehyde causes the formation of chemical cross-links so that the probe is covalently labeled with enzyme. After hybridization the detection solution is comprised of two detection reagents (DRI and II). DR-I decays to hydrogen peroxide, the substrate for peroxidase. Reduction of hydrogen peroxide by the enzyme is coupled to the light production reaction promoted by DR-II. This contains lumin.ol, which upon oxidation produces blue light. Hybridization is the re-annealing of a labeled single stranded DNA probe with a complementary sequence of genomic DNA on the filter.

About 300 ng of probe (plasmid digested with NotI and Xhol) was used for each hybridization reaction. The inserts were directly eluted from 0.7% LMP agarose and labeled with the labeling reagent. Pre-hybridization and hybridization (ECL™ GOLD buffer with 500 mM NaCl and 5% nonfat milk) were performed at 42° C. Blots were washed with primary wash buffer (0.5×SSC, 0.1% SDS, 6N Urea) for 2×30 min. at 43° C. and 2×10 min. with 2×SSC at room temperature with shaking. Then the blots were shocked with the detection reagent for two min.utes and then exposed to X-ray film.

FIG. 15 shows the PCR banding pattern of lignan genes in transgenic rice plants. Each plant carries a different number of genes.

C. LIGNAN PRODUCTION IN TRANSGENIC RICE

Lignan production was evaluated in transgenic rice plants which expressed lignan biosynthesis genes. Matairesinol concentrations in 142 transgenic seeds were measured, and analysis revealed that 21 transgenic plants produced seeds with elevated lignan levels.

### TABLE 6. MATAIRESINOL (NG/100MG) ING TRANSGENIC RICE SEEDS.

Total number of samples (R1 seeds) is 142. Category 5 is selected for advancing to R2/R3 and other subsequent generations (see Table 7).

TABLE 6
MATAIRESINOL (NG/100 MG) IN TRANSGENIC RICE SEEDS.
Total number of samples (R1 seeds) is 142. Category 5 is selected for
advancing to R2/R3 and other subsequent generations (see Table 7).

| Category | Number | Range | Mean | SD |
|---|---|---|---|---|
| 1 | 28 | 0.21-0.94 | 0.53 | 0.22 |
| 2 | 26 | 1.06-1.99 | 1.47 | 0.28 |
| 3 | 42 | 2.04-4.99 | 3.37 | 0.98 |
| 4 | 25 | 5.05-8.73 | 6.42 | 1.09 |
| 5 | 21 | 10.54-48.9 | 19.8 | 8.33 |
| Non- | 7 | 0.28-2.22 | 1.13 | 0.78 |
| transgenic | | | | |

TABLE 7
SELECTED LIGNAN PRODUCING LINES IN CATEGORY 5

| Line # | Matairesinol (ng/100 mg) |
|---|---|
| 4PE-20-1 | 19.26 |
| 4PE-25-1 | 15.92 |
| 4PE-57-1 | 32.6 |
| 4PE-59-1 | 16.88 |
| 4PE-80-1 | 10.54 |
| 4PE-94-1 | 13.31 |
| 4PE-102-1 | 20.88 |
| 4PE-103-1 | 19.71 |
| 4PE-104-1 | 20.12 |
| 4PE-112-1 | 19.62 |
| 4PE-115-1 | 24.51 |
| 4PE-131-1 | 12.26 |
| 4PE-147-1 | 15.84 |
| 4PE-212-1 | 17.5 |
| 4PE-213-1 | 26.22 |
| 4PE-230-1 | 15.12 |
| 4PE-245-1 | 18.64 |
| 4PE-256-1 | 19.29 |
| 4PE-264-1 | 17.06 |
| 4PE-265-1 | 48.90 |
| 4PE-348-1 | 12.63 |

In carrying out the analysis, rice R1 seeds harvested from transgenic plants were dissected into two halves. Embryo halves were kept and used to recover R1 transgenic plants. Endosperm halves were used to determin.e the concentration of lignan. About 7 half seeds from each transgenic plant were prepared to obtain over 100 mg of material for lignan assay (FIG. 13).

Embryo halves of transgenic rice seeds were sterilized in 50% v/v commercial bleach for 25 min. and washed with sterile water three times for 5 min. each. Sterilized half seeds were placed into test tube (one embryo/tube) containing MS media. The test tubes were then placed under strong light inorder to develop healthy plants from the embryos to obtain R2 seeds.

1. ENZYMATIC HYDROLYSIS

1 ml of distilled H₂O was added to a freeze-dried sample (100 mg) with the resulting slurry stirred at room temperature overnight. Hydrolysis is carried out in a glass tube in 2 ml of 0.3 M acetate buffer (pH 4.1) with 5000 Fishman units of Helix pomatia juice. Before hydrolysis, 2.5 mg ascorbic acid was added for the protection of labile metabolites. The sample was gently mixed with a Vortex mixer and incubated at 60° C. in a Dri-Block, for 2 h. After hydrolysis, the sample was cooled to room temperature, extracted with cold Et₂O (3 ml×5). The Et₂O extracts were combined and evaporated to dryness under nitrogen with gentle heating (45° C.) in a Dri-Block to give residue A, which was stored at −20° C. The water phase was then subjected to acid hydrolysis.

2. ACID HYDROLYSIS

To the water phase (2 ml) was added 6 M HCl (1 ml) to obtain a final concentration 2 M. The sample was mixed with Vortex mixer and incubated at 100° C. in the Dri-Bock. After 2.5 h, the sample was cooled to room temperature and the pH adjusted to 6 with 0.6 ml 10 M NaOH. Deuterium-labeled secoisolariciresinol (4) (67 ng), anhydrosecoisolariciresinol (5) (72 ng) and matairesinol (3) (55.2 ng) were next added (in CH₃OH, 20 μl) as internal standards for GC-MS analysis. The sample was then extracted with Et₂O (4 ml×5). This fraction was combined with residue A obtained after enzymatic hydrolysis. Finally, after evaporation of the solvent, the dry sample was re-dissolved in 1 ml CH₃OH.

3. CHROMATOGRAPHY ON DEAE-SEPHADEX OH

The free base form of DEAE-Sephadex was used to remove neutral steroids, etc. The DEAE-Sephadex was packed in CH₃OH in a Pasteur pipette (0.5 i.d.×3 cm) with a small piece of cotton in the bottom. The sample, dissolved in 1 ml CH₃OH, was loaded onto the DEAE-Sephadex (hydroxyl form) column. The column was eluted with 2.5 ml CH₃OH to first remove the neutral steroids. Lignans were obtained by eluting with 0.1 M HAc in CH₃OH (10 ml). This fraction was evaporated to dryness under N₂.

4. CHROMATOGRAPHY ON QAE-SEPHADEX AC.

QAE-Sephadex Ac was used to remove used to remove organic acids and many chromogens. The residue B, dissolved in 1 ml CH₃OH, was loaded onto the QAE-Sephadex (acetate form) column (0.5 i.d.×6 cm) equilibrated in CH₃OH. The column was eluted with 8 ml CH₃OH to give a fraction (9 ml) containing the lignans of interest. After evaporation to dryness under N₂, the residue was ready for TMS-derivatization.

5. PREPARATION OF TMS-DERIVATIVES

The dry samples were silylated by adding 100 μl of the silanization reagent (Tri-Sil, Pierce Co.). After incubation at 60° C. for 15 min., the solvent was evaporated to dryness under N₂. The resulting residue was then dissolved in 50 μl n-hexane containing 3% Silyl-8 (column conditioned). The n-hexane extracts were transferred to small vials with 1 μl of which submitted to GC-MS analysis.

6. GC-MS-SIM ANALYSIS

The GC-MS instrument consisted of a gas chromatograph (HP 6890 Series) and a HP 5973 quadrupole mass spectrometer equipped with a HP 7683 autoinjector. A 30 m×0.25 mm column (0.25 μm film thickness) was used (HP-5MS, crosslinked 5% PHME Siloxane). The carrier gas was helium (flow rate: 1.4 ml/min.). The initial temperature of the oven was 160° C., it was then increased by 30° C. every min.ute, to a final temperature of 280° C. and held at this temperature for 10 min.. The calculation of the results is carried out by comparing the peak area ratios of the ions for the plant-derived compounds and the deuterated internal standards d8-secoisolariciresinol (4), d6-anhydrosecoisolariciresinol (5) and d6-matairesinol (3) with the same peak area ratios of the calibrators used for preparing the standard curve. Standard curves were obtained with the lignans (1) - (3) using between 5 and 200 ng of each.

The embodiments of the invention in which an exclusive property or privilege is claimed are defined as follows:

1. A method of increasing the guaiacyl- (“G-”)lignan content in seeds of a monocot plant, comprising
selecting at least one protein or enzyme integral to the pathway leading to G-lignan formation
stably transformin.g a monocot plant with one or more chimeric gene constructs having a seed-specific transcriptional regulatory region operably linked to a nucleic acid sequence encoding said at least one protein or enzyme integral to the pathway leading to G-lignan formation.

2. The method according to claim 1 wherein said at least one protein or enzyme integral to the pathway leading to G-lignan formation is selected from the group consisting of (i) a dirigent protein (SEQ ID NO: 16), (ii) pinoresinol/lariciresinol reductase (SEQ ID NO: 20), (iii) secosisolari-ciresinol dehydrogenase (SEQ ID NO: 24), and (iv) laccase (SEQ ID NO:12).

3. The method according to claim 2 wherein (i) a dirigent protein (SEQ ID NO:16), (ii) pinoresinol/lariciresinol reductase (SEQ ID NO:20), (iii) secosisolari-ciresinol dehydrogenase (SEQ ID NO:24), and (iv) laccase (SEQ ID NO: 12), are expressed at the same time in seeds of a monocot plant.

4. The method according to claim 2 or 3 wherein the amount of (−)-secoisolariciresinol diglucoside or (−)-matairesinol accumulated in said seeds is greater than in seeds of an untransformed monocot plant.

5. The method according to claim 1 where said seed-specific transcript-ional regulatory region is derived from aleurone, pericarp, embryo or endosperm tissue.

6. The method according to claim 1 where said seed-specific transcript-ional regulatory region is induced during seed development and corresponds to an endosperm-specifc Gt-1 promoter.

7. The method according to claim 1 where said seed-specific transcriptional regulatory region is induced during seed development and corresponds to an aleurone-specifc Chi26 promoter.

8. The method of stably transforming a monocotyledonous plant with said one or more chimeric gene constructs according to claim 1 resulting in increased expression of said genes encoded by said one or more chimeric gene constructs.

9. The method according to claim 8 where the gene in said one or more chimeric gene constructs is selected from the group consisting of (i) a dirigent protein (SEQ ID NO: 15), (ii) pinoresinol/lariciresinol reductase (SEQ ID NO: 19), (iii) secosisolari-ciresinol dehydrogenase (SEQ ID NO: 23), and (iv) laccase (SEQ ID NO:11).

10. The method according to claim 2 where the nucleic acid sequence encoding said at least one protein or enzyme integral to the pathway leading to G-lignan formation has at least 90% sequence identity to a sequence selected from the group consisting of (i) a dirigent protein (SEQ ID NO:15), (ii) pinoresinol/lariciresinol reductase (SEQ ID NO: 19), (iii) secosisolari-ciresinol dehydrogenase (SEQ ID NO: 23), and (iv) laccase (SEQ ID NO:11).

11. The method according to claim 2 wherein the nucleic acid sequence encoding said protein or enzyme is capable of hybridizing under high stringency conditions and said protein or enzyme has substantially equivalent biological activity to the native protein or enzyme selected from the group consisting (i) a dirigent protein (SEQ ID NO:16), (ii) pinoresinol/lariciresinol reductase (SEQ ID NO:20), (iii) secosisolari-ciresinol dehydrogenase (SEQ ID NO: 24), and (iv) laccase (SEQ ID NO:12).

12. A transformed monocot plant produced by the method of claim 1.

13. The transformed monocot plant according to claim 15, capable of producing seeds where the amount of (−)-secoisolariciresinol diglucoside or (−)-matairesinol accumulated in the seeds of said transformed monocot plant is two or more times the amount detectable in seeds of an untransformed monocot plant.

14. The transformed monocot plant according to claim 15, capable of producing seeds where the amount of (−)-secoisolariciresinol diglucoside or (−)-matairesinol accumulated in the seeds of said transformed monocot plant five or more times the amount the amount detectable in seeds of an untransformed monocot plant.

15. A seed composition derived from a plant produced by the method of claim 1.

16. The seed composition according to claim 15, where the amount of (−)-secoisolariciresinol diglucoside or (−)-matairesinol accumulated in the seeds of said transformed monocot plant is two or more times the amount detectable in seeds of an untransformed monocot plant.

17. The seed composition according to claim 15, where the amount of (−)-secoisolariciresinol diglucoside or (−)-matairesinol accumulated in the seeds of said transformed monocot plant is five or more times the amount detectable in seeds of an untransformed monocot plant.

18. A library comprising seeds derived from one or more monocot plants produced by the method of claim 1.

19. A method of producing a progeny monocot plant by crossing one or more parent monocot plants produced by the method of claim 1 where the amount of G-lignans in the seeds of said progeny monocot plant resulting from said crossing is greater than the amount of G-lignans in the seeds of said parent monocot plant.

20. A G-lignan enriched seed composition for use as a food additive comprising a seed preparation derived from seeds of a transformed monocot plant wherein the amount of (−)-secoisolariciresinol diglucoside or (−)-matairesinol accumulated in said seeds is two or more times the amount detectable in seeds of an untransformed monocot plant.

21. The use of a G-lignan enriched seed composition according to claim 15 for use as a food additive.

22. The method of claim 1 wherein the monocot plant is a rice plant.

23. A transformed rice plant produced by the method of claim 1.

24. A seed composition derived from a rice plant produced by the method of claim 1.

25. A library comprising seeds derived from rice plants produced by the method of claim 1.
